SEQUENCE LISTING (1 sequence)

SEQ ID NO: 1 - DNA, 20 bp, Artificial Sequence
  Synthetic oligonucleotide
gcaaatgacagttcaagccc

(19) United States
(12) Patent Application Publication
(10) Pub. No.: US 2021/0205292 A1
(43) Pub. Date: Jul. 8, 2021

(54) INHIBITORS OF CREB-CBP INTERACTION FOR TREATMENT OF LEUKEMIA

(71) Applicant: The Board of Trustees of the Leland Stanford Junior University, Stanford, CA (US)

(72) Inventors: Kathleen Miho Sakamoto, Stanford, CA (US); Mark Smith, San Francisco, CA (US); Hee-Don Chae, Stanford, CA (US); Bryan Mitton, Redondo Beach, CA (US); Nicholas Raymond Cox, Seattle, WA (US)

(21) Appl. No.: 16/081,396

(22) PCT Filed: Mar. 10, 2017
(86) PCT No.: PCT/US2017/021959

§ 371 Date: Aug. 30, 2018

Related U.S. Application Data
(60) Provisional application No. 62/307,119, filed on Mar. 11, 2016.

(51) Int. Cl. A61K 31/4709

(52) U.S. Cl. CPC A61K 31/4709

(57) ABSTRACT

Compounds and methods are provided for inhibiting a CREB-CBP protein-protein interaction in a sample. In some cases, the method includes modulating transcription of CREB in a cell that overexpresses CREB. Also provided are methods of inhibiting the proliferation of a cancer cell. The subject CREB transcription inhibitor compounds include a substituted salicylamide or a prodrug thereof. Methods of alleviating symptoms associated with cancer (e.g., Acute Myeloid Leukemia (AML) or Acute Lymphomblastic Leukemia (ALL)) in a subject in need thereof are also provided. Pharmaceutical compositions including the subject compounds find use in treating cancer. The subject compounds may be formulated or provided to a subject in combination with a second agent, e.g. an anticancer agent.

### CROSS-REFERENCE

This application claims the benefit of U.S. Provisional Patent Application No. 62/307,119, filed Mar. 11, 2016, which application is incorporated herein by reference in its entirety.

### GOVERNMENT RIGHTS

This invention was made with Government support under contract HL075826 awarded by the National Institutes of Health. The Government has certain rights in the invention.

### INTRODUCTION

Acute Myelogenous Leukemia (AML) is associated with a 5-year overall survival of less than 50% despite the use of intensive chemotherapy regimens and hematopoietic stem cell transplantation. Cure rates for relapsed or refractory disease are less than 30%. Treatment for AML is itself associated with significant morbidity and mortality, and most patients who survive experience at least one serious treatment-related long-term complication.

### SUMMARY

Compounds and methods are provided for inhibiting a CREB-CBP protein-protein interaction in a sample. In some cases, the method includes modulating transcription of CREB in a cell that overexpresses CREB. Also provided are methods of inhibiting the proliferation of a cancer cell. The subject CREB transcription inhibitor compounds include a substituted salicylamide or a prodrug thereof. Methods of alleviating symptoms associated with cancer (e.g., a hematologic cancer such as Acute Myeloid Leukemia (AML) or Acute Lymphomblastic Leukemia (ALL)) in a subject in need thereof are also provided. Pharmaceutical compositions including the subject compounds find use in treating cancer. The subject compounds may be formulated or provided to a subject in combination with a second agent, e.g. an anticancer agent.

These and other advantages and features of the disclosure will become apparent to those persons skilled in the art upon reading the details of the compositions and methods of use, which are more fully described below.

### BRIEF DESCRIPTION OF THE FIGURES

The skilled artisan will understand that the drawings, described below, are for illustration purposes only. The drawings are not intended to limit the scope of the present teachings in any way.

FIG. 1, panels A-F, shows compound Compound A Binds to the KIX Domain of CBP and Blocks CREB-Dependent Gene Transcription. Panel A) The structure of Compound A and its inactive analog Analog B. Panel B) The native human KIX domain and two mutant proteins were expressed as a fusion protein with GST and subjected to Biacore analysis to assess Compound A binding characteristics. Mutation of Arginine-600 to Alanine reduced binding of Compound A by ˜45% at a concentration of 5 μM, while a KIX mutant lacking amino acids 586-602 reduced binding by ˜70%. Panel C) Binding model of Compound A to CBP KIX domain. Panel D) Split-Renilla luciferase assays with 293T cells treated with forskolin demonstrated Compound A is a direct inhibitor of CREB-CBP binding, with an IC₅₀ of approximately 3.2 μM. Panel E). Two KG-1 cell lines were generated in which luciferase was expressed either under the control of a CREB-driven promoter (CRE) or a CMV-driven promoter (CMV). These two cell lines were each treated with a range of Compound A or Analog B concentrations for 6 hours. Luciferase activity was significantly decreased following Compound A treatment at concentrations of 3, 10 and 30 μM. No statistical difference in luciferase activity was detected following Analog B treatment. Panel F) Compound A inhibits CREB/CBP association. HEK293 cells were transfected with a plasmid expressing CREB. Cells were treated with Compound A (A, 5 μM, lane 3) or DMSO vehicle (D) for 1 hour. Total lysates of transfected HEK293 cells were immunoprecipitated using anti-CBP antibody. Compound A (A, 5 μM) was added during anti-CBP Immunoprecipation process (lane 1). Immunoprecipitates (CBP-IP) and total lysates were analyzed by immunoblotting for phospho-CREB (p-CREB) and CBP.

FIG. 2, panels A-G, shows the efficacy of CREB Inhibition Depends on CREB Expression. Panel A) IC₅₀ values for 4 AML cells lines identically treated with Compound A are shown. Panel B) Western blot of CREB expression levels in 4 AML cell lines, run on non-contiguous lanes. Panel C) Western blot of CREB expression levels in KG-1 cells engineered to overexpress CBP or CREB, or in which CREB expression was reduced by shRNA. Panel D) Compound A dose-response data for the four KG-1 cell lines depicted in (C) following 48 hours of treatment. The IC₅₀ values were: CREB KD, 1.787 μM (white diamond); GFP, 1.040 μM (blue triangle); CREB OE, 0.687 μM (red square); CBP OE, 0.826 μM (grey circle). The same four cell lines treated with 30 μM the inactive analog Analog B showed no reduction in viability or proliferation rate (corresponding black symbols). Panel E) Six AML patient and three normal bone marrow samples were treated with 2 μM Compound A for 72 hours, and the percent of viable cells lost or gained compared to DMSO-treated cells is shown. Panel F) Western blot of CREB expression in ten AML patient and four normal marrow samples. Panel G) Methylcellulose colony assays of normal bone marrow progenitor cells treated with up to 50 μM Compound A

FIG. 3, panels A-G, demonstrates the specificity of CREB Inhibition. Panel A) The consensus sequence obtained following CREB ChIP-Seq, mapped against the canonical CRE element sequence. Panel B) The changes in relative expression and H3K27 acetylation following Compound A treatment are plotted for the 4680 CREB-bound genes identified on CREB ChIP-Seq. Panel C) Changes in H3K27 acetylation for CREB-bound and -unbound genes were averaged and plotted against base-pair distance to transcriptional start sites (TSS) following Compound A (yellow line) or DMSO (blue line) treatment. Non-CREB-bound genes show no significant change in H3K27 acetylation following Compound A treatment. Panel D) Heatmap of CREB binding and H3K27 acetylation relative to TSS in DMSO and Compound A-treated samples. H3K27 signal intensity, but not CREB binding signal intensity, decreased following Compound A. Panel E) Western blot of total and H3K27-specific histone acetylation following Compound A treatment following 6 or 24 hours of DMSO or Compound A treatment. Panel F) RT-PCR confirmed downregulation of CREB-bound genes identified on RNA-Seq following Compound A treatment of KG-1 cells for all genes shown, p<0.05. Panel G) RNA-Seq analysis demonstrates that the transcriptional activity of six CBP-bound transcription factors remain unchanged following Compound A treatment.

FIG. 4, panels A-E, demonstrates CREB inhibition in vivo. Panel A) Bioluminescent imaging revealed significantly less disease burden in mice treated with daily intravenous injections of Compound A on treatment days 10, 14 and 17 (DMSO-treated mice, left; Compound A-treated mice, right). Panel B) Kaplan-Meier curve analysis demonstrated a significant survival advantage in NSG mice treated with 2.3 mg/kg/day IV once a day Compound A compared to those treated with vehicle alone beginning one day after AML cell injection (p=0.002). Panel C) Kaplan-Meier curve analysis also demonstrated a survival advantage for mice given Compound A beginning 7 days after AML cell injection (p=0.021). Panel D) AML cell disease burden was reduced in Compound A-treated in both immediate (black bars) and delayed (grey bars) treatment groups, based on spleen weight and % GFP+ cells in bone marrow and spleen (* indicates p<0.05 versus DMSO-treated mice). Panel E) RT-PCR showed Compound A elicits the same transcriptional alterations in vivo as observed in vitro. Compound A treatment significantly reduced expression of all genes shown, p<0.05.

FIG. 5, panels A-E, illustrates that CREB inhibition in AML Cells Induces Apoptosis. Panel A) Flow cytometry demonstrated that HL-60 cells become apoptotic (c-PARP+) or die (aqua amine+) following 72 hours of treatment with 2 μM Compound A. Panel B) Caspase-3 activity is activated in response to Compound A treatment (* indicates p<0.05 compared to DMSO treatment). Panel C) RT-PCR showed Bcl-2 expression decreases at 72 hours after Compound A treatment in HL-60 cells. Panel D) Western blot analysis shows a decrease in Bcl-2 protein expression following 72 hours of treatment, no change in Bcl-XL expression, and an initial increase followed by a decrease in Mcl-1 expression in HL-60 cells. In KG-1 cells, Mcl-1 and Bcl-2 also showed decreased expression following Compound A treatment. Panel E) Heatmap representing expression of p-CREB (Ser133), total CREB (CREB) and Bcl-2 in four primary AML samples treated with DMSO or Compound A as analyzed by mass cytometry. Expression shown as Arcsin ratio to DMSO control (first column). Gated cell populations based on CD34 and/or CD38 as indicated above heatmap. Patient 96 and 186 demonstrate downregulation of Bcl-2 in all cell populations in response to Compound A (red box) as well as decreases in total CREB and p-CREB (yellow boxes). In contrast, patient 97 and 111 demonstrate activation of p-CREB in a cell specific manner (white box, blue box) as well as no effect on Bcl-2 expression in patient 111 (blue dashed box).

FIG. 6, panels A-C, shows that CREB Inhibition Induces Cell Cycle Arrest. Panel A) Cell cycle phase analysis of KG-1 cells treated with Compound A showed G₁/S transition block and delayed S-phase progression. Panel B) CyTOF analysis of AML patient samples also showed a reduction of cells in G₂ and S phase following Compound A treatment. Panel C) CREB-regulated genes important for cell cycle progression through G1/S and S were downregulated. Cyclin A1 and D1 expression was decreased following 12 hours of treatment, while Fra-1 and RFC-3 expression decreased following 48 hours of treatment.

FIG. 7, panels A-B, shows that compound Compound A is Synergistic with Daunomycin and Cytarabine. Isobolograms were generated for KG-1 cells treated with both Compound A and daunorubicin (A) or cytarabine (B) for 48 hours. The reduction in viable cell count was greater than predicted by calculated lines of equivalency, indicating that Compound A is synergistic with both of these agents.

FIG. 8 illustrates the viability of Primary AML cells and Normal Bone Marrow Progenitor Cells treated with compound Compound A. AML patient samples were cultured as described in Methods for 72 hours in the presence of 0.1% DMSO or 2 μM Compound A. Viable cells remaining after Compound A treatment as measured by Trypan blue exclusion assay are shown as a percent of cells present in DMSO-treated samples at the end of the treatment period. Cell viability in DMSO-treated samples either increased, or decreased by <10% after 72 hours in culture, similar to the response of normal bone marrow cells to Compound A. (Black bars, DMSO-treated AML samples; white bars, DMSO-treated normal bone marrow samples; grey bars, Compound A-treated samples).

FIG. 9, panels A-E, shows CREB Genomic Binding Site Characteristics and H3K27 Acetylation/Gene Expression Effects of Compound A. Panel A) Distribution of CREB-occupied CRE sites plotted against distance to gene transcription start sites (TSS) in DMSO-treated cells (left) and Compound A cells (right) shows that 90% of all CREB-binding sites are within 500 bp of TSS. Panel B) Distribution of identified CREB peaks across genetic elements. Panel C) RNA-Seq CREB-binding peaks (top) and H3K27 acetylation peaks (bottom) are shown for 3 genes. Compound A elicited reduced H2K27 acetylation at these gene loci, but not loss of CREB binding. Panel D) RT-PCR validation of transcriptional changes caused by Compound A of CREB-bound genes identified on RNA-Seq, performed in HL-60 cells. Compound A significantly reduced expression of all genes shown for both KG-1 and HL-60 AML cell lines, p<0.05. Panel E) Myb-driven gene expression was also examined in KG-1 and HL-60 cells under identical treatment conditions. No changes in gene expression were observed.

FIG. 10, panels A-D, illustrates the non-toxicity and pharmacodynamics of compound Compound A. Panel A) Human AML patient sample cells (#186) were injected (2×10⁶ cells) into 8 NSG mice, and mice were then treated with intravenous 0.1% DMSO or 2.3 mg/kg Compound A IV once daily. None of the mice treated for 80 days experienced any toxicity. Kaplan-Meier analysis showed a survival advantage for mice treated with Compound A. Panel B) To assess the half-life of Compound A, NSG mice were injected with Compound A (20 mg/kg/day IP), and three mice were sacrificed at 2, 4, 6 and 8 hours after injection for plasma analysis. Compound A plasma concentration was measured by quantitative mass spectrometry. Standard curve fitting reveals first order elimination of Compound A with a half-life of 4.3 hours. Panel C). Laboratory studies showed no difference between Compound A-treated mice and normal NSG mice with regard to bone marrow, liver or renal function. Numbers shown are averages of values from 3 mice SE. Panel D) Tissue histology showed no evidence of fibrosis or damage following Compound A treatment compared to normal NSG mice.

FIG. 11, panels A-D, illustrates ABT-737 Causes Apoptosis in HL-60 Cells; CyTOF Gating Scheme. Panel A) Treatment of HL-60 cells with the validated Bcl-2 inhibitor ABT-737 (50 nM) reduced cell viability after 48 hours of treatment, as measured by Trypan blue exclusion assay (*, p<0.05). Panel B) Flow cytometry demonstrated that HL-60 cells become apoptotic (c-PARP+) or die (aqua amine+) following 72 hours of treatment with 50 nM ABT-737. Panel C) Western blot analysis showed Bcl-2 expression is higher in HL-60 cells compared to KG-1 cells, and that CREB overexpression, but not CBP overexpression, increased Bcl-2 expression in KG-1 cells. Panel D) CyTOF gating scheme, used to perform analysis specifically on AML cells within AML patient marrow samples.

FIG. 12, panels A-D, illustrates the results of CyTOF Phenotyping and Compound A Combination Studies. Panel A) CyTOF analysis of primary AML patient bone marrow samples showed that reduced activation of ERK and AKT after 72 hours of Compound A treatment was associated with reduced phosphorylation of CREB, while AKT activation appeared to correlate with increased CREB phosphorylation. Panel B) (bottom) HL-60 cells treated with Compound A for 24 hours showed an increase in CREB phosphorylation but not unphosphorylated CREB levels in a concentration dependent manner. (top) The use of specific, validated kinase inhibitors (BI-D1870 for RSK1-4, 5 μM; SB202190 for ERK1/2, 10 μM; U0126 for p38, 10 μM) identified that inhibition of either ERK1/2 or RSK kinases, but not the p38 kinase, blocked compensatory CREB phosphorylation following Compound A treatment in these cells. Panel C) Treatment of HL-60 cells for 48 hours with combinations of Compound A and BI-D1870 or SB202190, but not U0126, showed additive effects. Viability for Compound A treatment alone was 87.4±7.3%, versus 55.3±3.4% and 49.5±2.3% when combined with ERK and RSK inhibitors, respectively (* indicates p<0.05 compared to cells treated with Compound A alone). Panel D) Compound A inhibits CREB phosphorylation in the presence of serum. HL-60 cells were serum-starved then stimulated with serum. CREB phosphorylation decreases after 48 hours of Compound A treatment.

FIG. 13, panels A-C, illustrates Cell Cycle Analysis and Cyclin Dependent Kinase Expression Following Compound A Treatment. Panel A) Flow cytometry revealed that treatment of KG-1 AML cells with Compound A resulted in G1 arrest, most pronounced 12 hours following release from nocodazole block. Panel B) Combined CREB ChIP-Seq and RNA-Seq analysis showed that many cyclin-dependent kinases are bound by CREB, and their expression decreased following treatment with Compound A. Panel C) RT-PCR confirmation of transcriptional downregulation of a set of cyclin-dependent kinases and related genes in HL-60 and KG-1 cells. All genes shown were significantly downregulated, p<0.05.

### DEFINITIONS

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of embodiments of the present disclosure.

It must be noted that as used herein and in the appended claims, the singular forms “a”, “and”, and “the” include plural referents unless the context clearly dictates otherwise. Thus, for example, reference to “a compound” includes not only a single compound but also a combination of two or more compounds, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The terms “active agent,” “antagonist”, “inhibitor”, “drug” and “pharmacologically active agent” are used interchangeably herein to refer to a chemical material or compound which, when administered to an organism (human or animal) induces a desired pharmacologic and/or physiologic effect by local and/or systemic action.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease); (b) inhibiting the disease, i.e., arresting its development; and (c) relieving the disease, i.e., causing regression of the disease (e.g., reduction in titers of cancer cells).

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the term “sample” relates to a material or mixture of materials, in some cases in liquid form, containing one or more analytes of interest. In some embodiments, the term as used in its broadest sense, refers to any plant, animal or bacterial material containing cells or producing cellular metabolites, such as, for example, tissue or fluid isolated from an individual (including without limitation plasma, serum, cerebrospinal fluid, lymph, tears, saliva and tissue sections) or from in vitro cell culture constituents, as well as samples from the environment. The term “sample” may also refer to a “biological sample”. As used herein, the term “a biological sample” refers to a whole organism or a subset of its tissues, cells or component parts (e.g. body fluids, including, but not limited to, blood, mucus, lymphatic fluid, synovial fluid, cerebrospinal fluid, saliva, amniotic fluid, amniotic cord blood, urine, vaginal fluid and semen). A “biological sample” can also refer to a homogenate, lysate or extract prepared from a whole organism or a subset of its tissues, cells or component parts, or a fraction or portion thereof, including but not limited to, plasma, serum, spinal fluid, lymph fluid, the external sections of the skin, respiratory, intestinal, and genitourinary tracts, tears, saliva, milk, blood cells, tumors and organs. In certain embodiments, the sample has been removed from an animal or plant. Biological samples may include cells. The term “cells” is used in its conventional sense to refer to the basic structural unit of living organisms, both eukaryotic and prokaryotic, having at least a nucleus and a cell membrane. In certain embodiments, cells include prokaryotic cells, such as from bacteria. In other embodiments, cells include eukaryotic cells, such as cells obtained from biological samples from animals, plants or fungi.

As used herein, the terms “determining,” “measuring,” “assessing,” and “assaying” are used interchangeably and include both quantitative and qualitative determinations.

A “therapeutically effective amount”, “effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of a compound (e.g., an aminopyrimidine compound, as described herein) calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

As used herein, the phrase “having the formula” or “having the structure” is not intended to be limiting and is used in the same way that the term “comprising” is commonly used. The term “independently selected from” is used herein to indicate that the recited elements, e.g., R groups or the like, can be identical or different.

As used herein, the terms “may,” “optional,” “optionally,” or “may optionally” mean that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

As used herein, the term “electron withdrawing group” or “EWG” refers to a substituent group that withdraws electron density from atoms to which it is attached towards itself, e.g., via resonance or inductive effects. Any convenient electron withdrawing groups can be utilized in the subject compounds. EWGs of interest include, but are not limited to, trifluoromethyl, nitro, cyano, sulfonyl group, sulfonate, ammonium, carbonyl groups, carboxy, keto, aldehyde, ester, and the like. Electron donating groups have an opposite effect. Electron donating groups of interest include, but are not limited to, alkyl, amino, alkoxy, hydroxy and substituted versions thereof.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

The term “alkyl” as used herein refers to a branched or unbranched saturated hydrocarbon group (i.e., a mono-radical) typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although not necessarily, alkyl groups herein may contain 1 to about 18 carbon atoms, and such groups may contain 1 to about 12 carbon atoms. The term “lower alkyl” intends an alkyl group of 1 to 6 carbon atoms. “Substituted alkyl” refers to alkyl substituted with one or more substituent groups, and this includes instances wherein two hydrogen atoms from the same carbon atom in an alkyl substituent are replaced, such as in a carbonyl group (i.e., a substituted alkyl group may include a —C(═O)— moiety). The terms “heteroatom-containing alkyl” and “heteroalkyl” refer to an alkyl substituent in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “substituted alkyl” is meant to include an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R and R″ may be the same or different and are chosen from hydrogen, optionally substituted alkyl, cycloalkyl, alkenyl, cycloalkenyl, alkynyl, aryl, heteroaryl and heterocyclic.

The term “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein may contain 2 to about 18 carbon atoms, and for example may contain 2 to 12 carbon atoms. The term “lower alkenyl” intends an alkenyl group of 2 to 6 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Substituents identified as “C1-C6 alkoxy” or “lower alkoxy” herein may, for example, may contain 1 to 3 carbon atoms, and as a further example, such substituents may contain 1 or 2 carbon atoms (i.e., methoxy and ethoxy).

The term “substituted alkoxy” refers to the groups substituted alkyl-O—, substituted alkenyl-O-substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “aryl” as used herein, and unless otherwise specified, refers to an aromatic substituent generally, although not necessarily, containing 5 to 30 carbon atoms and containing a single aromatic ring or multiple aromatic rings that are fused together, directly linked, or indirectly linked (such that the different aromatic rings are bound to a common group such as a methylene or ethylene moiety). Aryl groups may, for example, contain 5 to 20 carbon atoms, and as a further example, aryl groups may contain 5 to 12 carbon atoms. For example, aryl groups may contain one aromatic ring or two or more fused or linked aromatic rings (i.e., biaryl, aryl-substituted aryl, etc.). Examples include phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. Aryl is intended to include stable cyclic, heterocyclic, polycyclic, and polyheterocyclic unsaturated C₃-C₁₄ moieties, exemplified but not limited to phenyl, biphenyl, naphthyl, pyridyl, furyl, thiophenyl, imidazoyl, pyrimidinyl, and oxazoyl; which may further be substituted with one to five members selected from the group consisting of hydroxy, C₁-C₈ alkoxy, C₁-C₈ branched or straight-chain alkyl, acyloxy, carbamoyl, amino, N-acylamino, nitro, halogen, trifluoromethyl, cyano, and carboxyl (see e.g. Katritzky, Handbook of Heterocyclic Chemistry). If not otherwise indicated, the term “aryl” includes unsubstituted, substituted, and/or heteroatom-containing aromatic substituents.

The term “aralkyl” refers to an alkyl group with an aryl substituent, and the term “alkaryl” refers to an aryl group with an alkyl substituent, wherein “alkyl” and “aryl” are as defined above. In general, aralkyl and alkaryl groups herein contain 6 to 30 carbon atoms. Aralkyl and alkaryl groups may, for example, contain 6 to 20 carbon atoms, and as a further example, such groups may contain 6 to 12 carbon atoms.

The term “alkylene” as used herein refers to a di-radical alkyl group. Unless otherwise indicated, such groups include saturated hydrocarbon chains containing from 1 to 24 carbon atoms, which may be substituted or unsubstituted, may contain one or more alicyclic groups, and may be heteroatom-containing. “Lower alkylene” refers to alkylene linkages containing from 1 to 6 carbon atoms. Examples include, methylene (—CH₂—), ethylene (—CH₂CH₂—), propylene (—CH₂CH₂CH₂—), 2-methylpropylene (—CH₂—CH(CH₃)—CH₂—), hexylene (—(CH₂)₆—) and the like.

Similarly, the terms “alkenylene,” “alkynylene,” “arylene,” “aralkylene,” and “alkarylene” as used herein refer to di-radical alkenyl, alkynyl, aryl, aralkyl, and alkaryl groups, respectively.

The term “amino” is used herein to refer to the group —NRR′ wherein R and R′ are independently hydrogen or nonhydrogen substituents, with nonhydrogen substituents including, for example, alkyl, aryl, alkenyl, aralkyl, and substituted and/or heteroatom-containing variants thereof.

The terms “halo” and “halogen” are used in the conventional sense to refer to a chloro, bromo, fluoro or iodo substituent.

“Carboxyl,” “carboxy” or “carboxylate” refers to —CO₂H or salts thereof.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO— heteroaryl, —SO₂-alkyl, —SO₂-substituted alkyl, —SO₂-aryl and —SO₂-heteroaryl.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the terms “heterocyclic” or “heterocycle” refer to a cyclic substituent that is heteroatom-containing, the terms “heteroaryl” and “heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, furyl, pyrimidinyl, imidazolyl, 1,2,4-triazolyl, tetrazolyl, etc., and examples of heteroatom-containing alicyclic groups are pyrrolidino, morpholino, piperazino, piperidino, tetrahydrofuranyl, etc.

As used herein, the terms “Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 15 ring atoms, including 1 to 4 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO— alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Hydrocarbyl” refers to univalent hydrocarbyl radicals containing 1 to about 30 carbon atoms, including 1 to about 24 carbon atoms, further including 1 to about 18 carbon atoms, and further including about 1 to 12 carbon atoms, including linear, branched, cyclic, saturated and unsaturated species, such as alkyl groups, alkenyl groups, aryl groups, and the like. A hydrocarbyl may be substituted with one or more substituent groups. The term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation, functional groups, and the hydrocarbyl moieties C1-C24 alkyl (including C1-C18 alkyl, further including C1-C12 alkyl, and further including C1-C6 alkyl), C2-C24 alkenyl (including C2-C18 alkenyl, further including C2-C12 alkenyl, and further including C2-C6 alkenyl), C2-C24 alkynyl (including C2-C18 alkynyl, further including C2-C12 alkynyl, and further including C2-C6 alkynyl), C5-C30 aryl (including C5-C20 aryl, and further including C5-C12 aryl), and C6-C30 aralkyl (including C6-C20 aralkyl, and further including C6-C12 aralkyl). The above-mentioned hydrocarbyl moieties may be further substituted with one or more functional groups or additional hydrocarbyl moieties such as those specifically enumerated. Unless otherwise indicated, any of the groups described herein are to be interpreted as including substituted and/or heteroatom-containing moieties, in addition to unsubstituted groups.

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

By the term “functional groups” is meant chemical groups such as halo, hydroxyl, sulfhydryl, C1-C24 alkoxy, C2-C24 alkenyloxy, C2-C24 alkynyloxy, C5-C20 aryloxy, acyl (including C2-C24 alkylcarbonyl (—CO-alkyl) and C6-C20 arylcarbonyl (—CO-aryl)), acyloxy (—O-acyl), C2-C24 alkoxycarbonyl (—(CO)—O-alkyl), C6-C20 aryloxycarbonyl (—(CO)—O-aryl), halocarbonyl (—CO)—X where X is halo), C2-C24 alkylcarbonato (—O—(CO)—O-alkyl), C6-C20 arylcarbonato (—O—(CO)—O-aryl), carboxy (—COOH), carboxylato (—COO—), carbamoyl (—(CO)—NH2), mono-substituted C1-C24 alkylcarbamoyl (—(CO)—NH(C1-C24 alkyl)), di-substituted alkylcarbamoyl (—(CO)—N(C1-C24 alkyl)2), mono-substituted arylcarbamoyl (—(CO)—NH-aryl), thiocarbamoyl (—(CS)—NH2), carbamido (—NH—(CO)—NH2), cyano (—C≡N), isocyano (—N+≡C—), cyanato (—O—C≡N), isocyanato (—O—N+≡C—), isothiocyanato (—SC≡N), azido (—N═N+═N—), formyl (—(CO)—H), thioformyl (—(CS)—H), amino (—NH2), mono- and di-(C1-C24 alkyl)-substituted amino, mono- and di-(C5-C20 aryl)-substituted amino, C2-C24 alkylamido (—NH—(CO)-alkyl), C5-C20 arylamido (—NH—(CO)-aryl), imino (—CR═NH where R=hydrogen, C1-C24 alkyl, C5-C20 aryl, C6-C20 alkaryl, C6-C20 aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO2), nitroso (—NO), sulfo (—SO₂—OH), sulfonato (—SO₂—O—), C1-C24 alkylsulfanyl (—S-alkyl; also termed “alkylthio”), arylsulfanyl (—S-aryl; also termed “arylthio”), C1-C24 alkylsulfinyl (—(SO)-alkyl), C5-C20 arylsulfinyl (—(SO)-aryl), C1-C24 alkylsulfonyl (—SO2-alkyl), C5-C20 arylsulfonyl (—SO₂-aryl), phosphono (—P(O)(OH)₂), phosphonato (—P(O)(O—)₂), phosphinato (—P(O)(O—)), phospho (—PO₂), and phosphino (—PH₂), mono- and di-(C1-C24 alkyl)-substituted phosphino, mono- and di-(C5-C20 aryl)-substituted phosphine. In addition, the aforementioned functional groups may, if a particular group permits, be further substituted with one or more additional functional groups or with one or more hydrocarbyl moieties such as those specifically enumerated above.

By “linking” or “linker” as in “linking group,” “linker moiety,” etc., is meant a bivalent radical moiety that connects two groups via covalent bonds. Examples of such linking groups include alkylene, alkenylene, alkynylene, arylene, alkarylene, aralkylene, and linking moieties containing functional groups including, without limitation: amido (—NH—CO—), ureylene (—NH—CO—NH—), imide (—CO—NH—CO—), epoxy (—O—), epithio (—S—), epidioxy (—O—O—), carbonyldioxy (—O—CO—O—), alkyldioxy (—O—(CH2)n-O—), epoxyimino (—O—NH—), epimino (—NH—), carbonyl (—CO—), etc. Any convenient orientation and/or connections of the linkers to the linked groups may be used.

When the term “substituted” appears prior to a list of possible substituted groups, it is intended that the term apply to every member of that group. For example, the phrase “substituted alkyl and aryl” is to be interpreted as “substituted alkyl and substituted aryl.”

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR, —NR⁸⁰R⁸⁰, trihalomethyl, —CN, —OCN, —SCN, —NO, —NO₂, ═N₂, —N₃, —SO₂R⁷⁰, —SO₂O⁻ M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰) R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —N R⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

In certain embodiments, a substituent may contribute to optical isomerism and/or stereo isomerism of a compound. Salts, solvates, hydrates, and prodrug forms of a compound are also of interest. All such forms are embraced by the present disclosure. Thus the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C.

Definitions of other terms and concepts appear throughout the detailed description below.

### DETAILED DESCRIPTION OF THE EMBODIMENTS

As summarized above, compounds and methods are provided for inhibiting a CREB-CBP protein-protein interaction in a sample. In some cases, the method includes modulating transcription of CREB in a cell that overexpresses CREB. Also provided are methods of inhibiting the proliferation of a cancer cell. The subject CREB transcription inhibitor compounds include a substituted salicylamide or a prodrug thereof. Methods of alleviating symptoms associated with cancer (e.g., Acute Myeloid Leukemia (AML) or Acute Lymphomblastic Leukemia (ALL)) in a subject in need thereof are also provided. Pharmaceutical compositions including the subject compounds find use in treating cancer. The subject compounds may be formulated or provided to a subject in combination with a second agent, e.g. an anticancer agent.

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

All patents and publications, including all sequences disclosed within such patents and publications, referred to herein are expressly incorporated by reference.

### CREB-CREB Binding Protein (CBP) Protein-Protein Interaction

The transcription factor CREB (cAMP Response-Element Binding Protein) is a critical regulator of the growth and survival of AML cells. Elevated CREB expression is observed in 60% of AML patients, and this is associated with a significantly worse prognosis and an increased risk of relapse compared to patients with basal CREB expression, independent of other negative prognostic factors. CREB overexpression in AML cells augments their growth rate and confers resistance to apoptosis in vitro. Conversely, CREB knockdown inhibited AML cell proliferation and induced apoptosis, but had no toxicity to normal hematopoietic stem cells in mouse transduction/transplantation assays. CREB is associated with a more aggressive form of AML, yet is not required for normal hematopoietic stem cell function. Therefore, inhibition of CREB function can represent an effective, targeted approach to AML therapy and treatment of a variety of other cancers.

CREB binds genomic DNA at thousands of loci possessing the consensus CREB DNA-binding site, termed the cAMP Response Element or ‘CRE’ site. Initiation of CREB-driven transcription at these loci requires that CREB recruits and binds a co-activator, the histone acetyltransferase CREBBinding Protein (CBP). This interaction triggers local histone acetylation and subsequent recruitment of the RNA polymerase transcriptional machinery to the promoter. Described herein is a method to disrupt the critical protein-protein interaction between CREB and its required co-activator to disrupt CREB-driven transcription.

The precise molecular interactions that mediate CREB-CBP binding have been resolved by NMR spectroscopy. The present disclosure provides a method of targeting the CREB/CBP co-activator interaction in AML cells. In some cases, the method provides low or substantially no toxicity to normal cells. To date, efforts to develop targeted cancer therapies have largely focused on catalytic-site inhibition of proteins with enzymatic activity, such as kinases or histone deacetylases. The use of small molecules to inhibit protein-protein interactions, especially transcription factors, presents unique challenges, as some otherwise promising targets are considered “undruggable”. The present disclosure demonstrates that the subject compounds can disrupt the CREB-CBP interaction in AML cells and elicit an array of ontarget transcriptional alterations. Disruption of CREB-driven transcription results in AML cell apoptosis and cell cycle arrest. Notably, the subject compounds exhibit little to no toxicity to normal hematopoietic cells or animals, even when treated with doses significantly greater than that necessary to kill AML cells. These results suggest that CREB/CRB inhibition represents a approach for treatment of a variety of cancers, including hematologic malignancies such as AML and Acute Lymphomblastic Leukemia (ALL).

The present disclosure provides compounds and method for inhibiting a CREB-CBP protein-protein interaction. In some embodiments, the inhibitor compound specifically binds the KIX domain of CREB Binding Protein (CBP), thereby inhibiting the interaction between CREB and CBP. Also provided are methods for modulating transcription of CREB in a cell that overexpresses CREB. The subject inhibitor compounds and methods find use in a variety of applications in which inhibition of a CREB-CBP protein-protein interaction is desired. Also provided are pharmaceutical compositions that include the subject inhibitor compounds, where a compound of the present disclosure can be formulated with a pharmaceutically acceptable excipient. Formulations may be provided in a unit dose, where the dose provides an amount of the compound effective to achieve a desired result, including without limitation inhibition of the protein-protein interaction, or modulation of CREB transcription.

### CREB-CBP Inhibitor Compounds

As summarized above, aspects of the present disclosure include CREB-CBP inhibitor compounds. In some cases, the compounds include a salicylamide core structure. The aryl rings of the core structure may include various particular combinations of further substituents. In some embodiments, prodrug forms of the salicylamide compounds are utilized, e.g., acyl or phosphate ester derivatives of any one of the compounds described herein which can be hydrolyzed in situ to release the salicylamide compound of interest. Exemplary compounds are set forth in the following structures and formulae.

In some cases, the subject compound is described by the structure of formula (I):

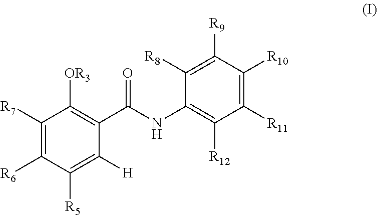

wherein:

R₃ is selected from H and a promoiety (e.g., an acyl, substituted acyl or a phosphate ester);

R₈, R₉, R₁₀, R₁₁ and R₁₂ are independently selected from H, halogen, alkyl, substituted alkyl, alkoxy, substituted alkoxy, an electron withdrawing group (e.g., cyano, nitro, trifluoromethyl, etc), phenyl, substituted phenyl, substituted amino, carboxy ester (e.g., CO₂R where R is alkyl or substituted alkyl); and

R₅, R₆ and R₇ are independently selected from H, F, Cl, Br, alkyl, substituted alkyl, aryl, substituted aryl, heteroaryl, substituted heteroaryl, an electron withdrawing group (e.g., cyano, nitro, trifluoromethyl, etc), alkoxy and substituted alkoxy, wherein optionally R₆ and R₇ or R₅ and R₆ are cyclically linked to form a fused aryl or heteroaryl ring which is optionally further substituted;

or a salt thereof, or a solvate, hydrate or prodrug form thereof.

In some cases, the subject compound is described by the structure of one of formulae (II)-(IV):

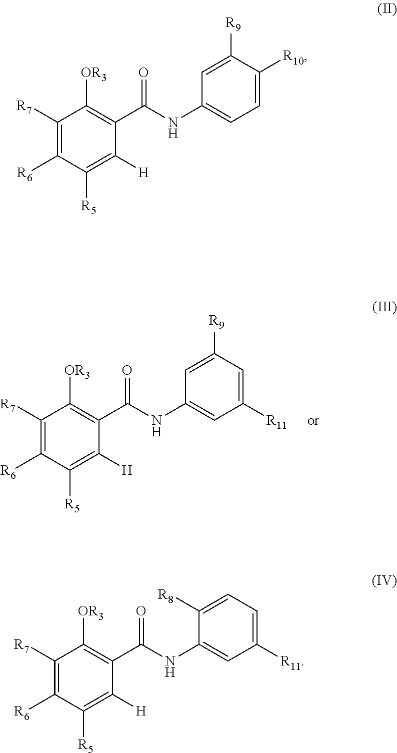

In certain instances, the compound is of formula (II). In certain instances, the compound is of formula (III). In certain instances, the compound is of formula (IV).

In some cases, the subject compound is described by one of formulae (V)-(VIII):

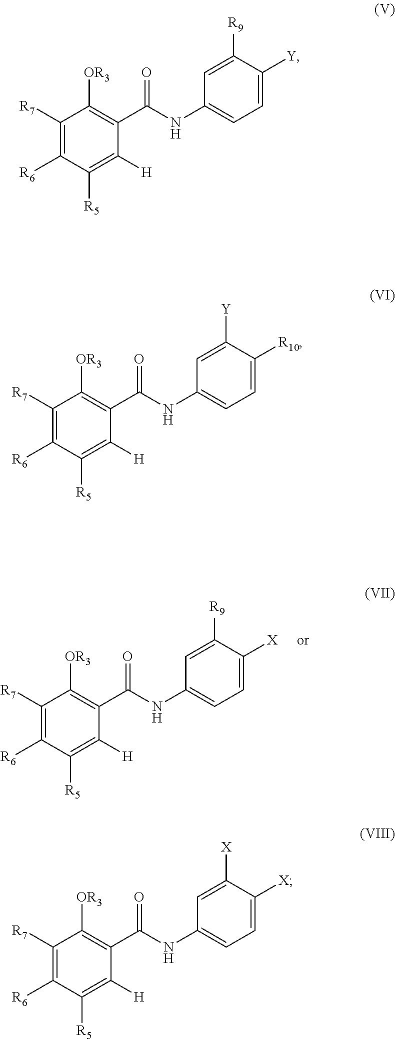

wherein: Y is an electron withdrawing group; and X is a halogen. In some cases of formulae (V)-(VIII), X is Cl. In some cases of formulae (V)-(VIII), X is F. In some cases of formulae (V)-(VIII), X is Br. In some cases of formulae (V)-(VIII), Y is cyano. In some cases of formulae (V)-(VIII), Y is nitro. In some cases of formulae (V)-(VIII), Y is trifluoromethyl. In certain instances, the compound is of formula (V). In certain instances, the compound is of formula (VI). In certain instances, the compound is of formula (VII). In certain instances, the compound is of formula (VIII).

In some cases, the subject compound is described by one of formulae (IX)-(XII):

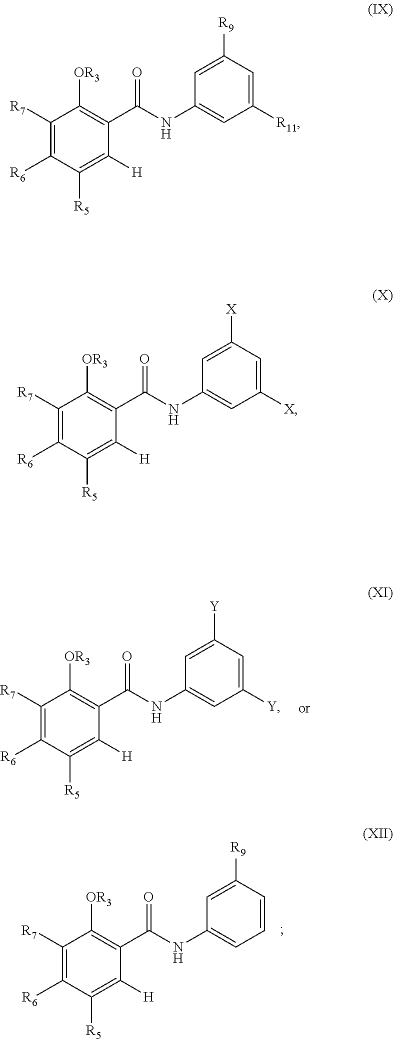

wherein: Y is an electron withdrawing group; and X is a halogen. In some cases of formulae (IX)-(XII), X is Cl. In some cases of formulae (IX)-(XII), X is F. In some cases of formulae (IX)-(XII), X is Br. In some cases of formulae (IX)-(XII), Y is cyano. In some cases of formulae (IX)-(XII), Y is nitro. In some cases of formulae (IX)-(XII), Y is trifluoromethyl. In certain instances, the compound is of formula (IX). In certain instances, the compound is of formula (X). In certain instances, the compound is of formula (XI). In certain instances, the compound is of formula (XII).

In some cases, the subject compound is described by one of formulae (XIII)-(XV):

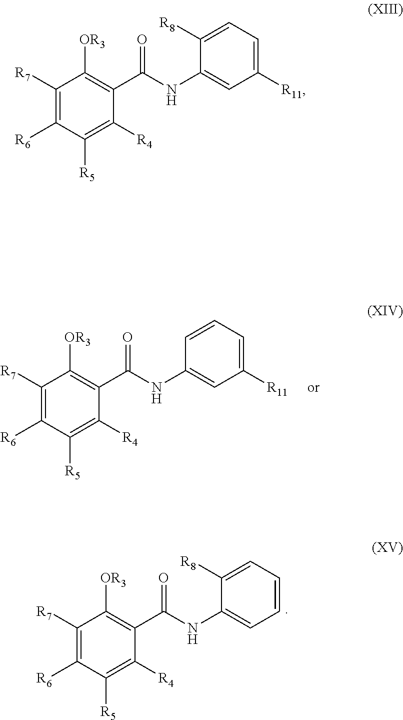

In some cases, the subject compound is described by formula (XVI):

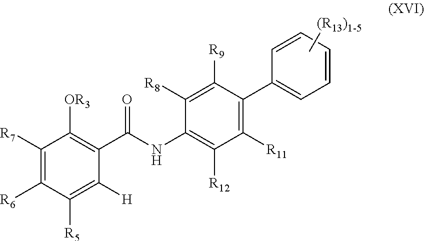

wherein each R₁₃ is independently selected from H, halogen, alkyl, substituted alkyl, alkoxy, substituted alkoxy, hydroxy, cyano, nitro, aryl, substituted aryl, heterocycle, substituted heterocycle, heteroaryl, substituted heteroaryl, amino, substituted amino, carboxy, carboxy ester (e.g., CO₂R where R is alkyl or substituted alkyl), sulfonyl, sulfonate, sulfonamide and substituted sulfonamide. In some cases, each R₁₃ is H. In certain instances, the compound is of formula (XIII). In certain instances, the compound is of formula (XIV). In certain instances, the compound is of formula (XV). In certain instances, the compound is of formula (XVII).

In some embodiments of formula (I)-(XVI), R₅ is halogen. In certain embodiments of formula (I)-(XVI), R₅ is Cl. In certain embodiments of formula (I)-(XVI), R₅ is Br. In certain embodiments of formula (I)-(XVI), R₅ is F. In some embodiments of formula (I)-(XVI), R₅ is an electron withdrawing group (e.g., CN, NO₂ or CF₃). In certain embodiments of formula (I)-(XVI), R₅ is cyano. In certain embodiments of formula (I)-(XVI), R₅ is nitro. In certain embodiments of formula (I)-(XVI), R₅ is CF₃.

In some embodiments of formula (I)-(XVI), R₆ and R₇ are each hydrogen. In some embodiments of formula (I)-(XVI), R₆ and R₇ are each hydrogen and R₅ is halogen (e.g., Cl or F).

In some embodiments of formula (I)-(XVI), R₆ is halogen. In certain embodiments of formula (I)-(XVI), R₆ is Cl. In certain embodiments of formula (I)-(XVI), R₆ is Br. In certain embodiments of formula (I)-(XVI), R₆ is F.

In some embodiments of formula (I)-(XVI), R₆ is an electron withdrawing group. In certain embodiments of formula (I)-(XVI), R₆ is cyano. In certain embodiments of formula (I)-(XVI), R₆ is nitro. In certain embodiments of formula (I)-(XVI), R₆ is CF₃. In some embodiments of formula (I)-(XVI), R₅ and R₇ are each hydrogen. In some embodiments of formula (I)-(XVI), R₅ and R₇ are each hydrogen and R₆ is halogen (e.g., Cl or F). In some embodiments of formula (I)-(XVI), R₅ and R₇ are each hydrogen and R₆ is an electron withdrawing group (e.g., CN, NO₂ or CF₃).

In some embodiments of formula (I)-(XVI), R₇ is halogen. In certain embodiments of formula (I)-(XVI), R₆ is Cl. In certain embodiments of formula (I)-(XVI), R₆ is Br. In certain embodiments of formula (I)-(XVI), R₆ is F. In some embodiments of formula (I)-(XVI), R₅ and R₆ are each hydrogen. In certain embodiments of formula (I)-(XVI), R₅ and R₆ are each hydrogen and R₇ is halogen (e.g., Cl, Br or F).

In some embodiments of formula (I)-(XVI), R₅ or R₆ is an aryl, a substituted aryl, a heteroaryl or a substituted heteroaryl. In some instances, one and only one of R₅ and R₆ is an aryl, a substituted aryl, a heteroaryl or a substituted heteroaryl and the other of R₅ and R₆ is hydrogen. In certain instances, R₅ or R₆ is a phenyl or substituted phenyl. In certain instances, R₅ or R₆ is a heteroaryl or substituted heteroaryl. In certain instances, R₅ or R₆ is a pyridyl (e.g., a 2-pyridyl, a 3-pyridyl or a 4-pyridyl) or substituted pyridyl. In certain instances, R₅ or R₆ is a pyrimidinyl (e.g., a 5-pyrimidinyl) or a substituted pyrimidinyl. In some embodiments, the inhibitor has one of formulae (XVII)-(XVIIIa):

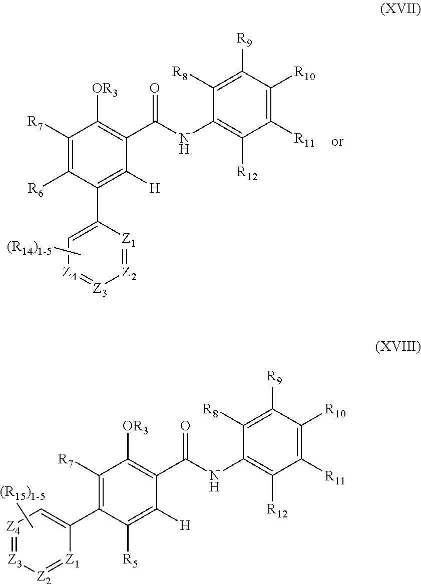

wherein:

each Z₁-Z₄ is independently CR₁₄, CR₁₅ or N, with the proviso that 0, 1 or 2 of the Z₁-Z₄ in the compound is CR₁₄ or CR₁₅; and

each R₁₄ and R₁₅ is independently selected from H, halogen, alkyl, substituted alkyl, alkoxy, substituted alkoxy, hydroxy, cyano, nitro, aryl, substituted aryl, heterocycle, substituted heterocycle, heteroaryl, substituted heteroaryl, amino, substituted amino, carboxy, carboxy ester (e.g., CO₂R where R is alkyl or substituted alkyl), sulfonyl, sulfonate, sulfonamide and substituted sulfonamide. In certain instances of formulae (XVII) and (XVIII), Z₁ is N and Z₂—Z₄ are independently CR₁₄ or CR₁₅. In certain instances of formulae (XVII) and (XVIII), Z₂ is N and Z₁ and Z₃—Z₄ are independently CR₁₄ or CR₁₅. In certain instances of formulae (XVII) and (XVIII), Z₃ is N and Z₄ and Z₁-Z₂ are independently CR₁₄ or CR₁₅. In certain instances of formulae (XVII) and (XVIII), Z₂ and Z₄ are N and Z₁ and Z₃ are independently CR₁₄ or CR₁₅. In certain instances of formulae (XVII) and (XVIII), Z₁-Z₄ are each independently CR₁₄ or CR₁₅. In certain embodiments of formula (XVII) and (XVIII), the compound is also a compound of one of formulae (II)-(XII).

In some embodiments of formula (XVIII), the inhibitor has the structure of formula (XVIIa) or (XVIIIa):

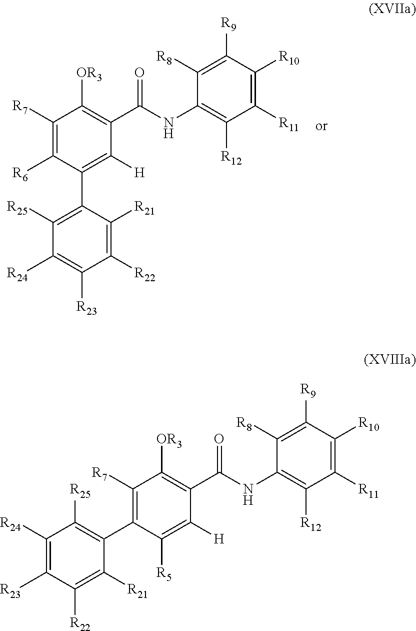

wherein R₂₁-R₂₅ are independently selected from H, halogen, alkyl, substituted alkyl, alkoxy, substituted alkoxy, hydroxy, cyano, nitro, aryl, substituted aryl, heterocycle, substituted heterocycle, heteroaryl, substituted heteroaryl, amino, substituted amino, carboxy, carboxy ester (e.g., CO₂R where R is alkyl or substituted alkyl), sulfonyl, sulfonate, sulfonamide and substituted sulfonamide. In certain embodiments of formula (XVIIa) and (XVIIIa), the compound is also a compound of one of formulae (II)-(XII). In certain cases, the compound has the structure of one of the compounds of Table 1.

TABLE 1
Exemplary compounds of Formula (XVIIIa):

(XVIIIa)

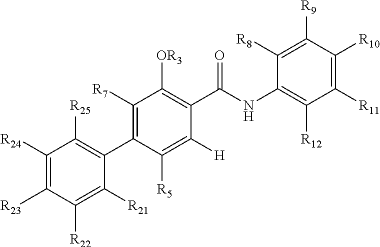

| Compound | R₃ | R₅ | R₇ | R₈ | R₉ | R₁₀ | R₁₁ | R₁₂ | R₂₁ | R₂₂ | R₂₃ | R₂₄ | R₂₅ |
|---|---|---|---|---|---|---|---|---|---|---|---|---|---|
| 101 | H | H | H | H | H | CN | H | H | H | H | H | H | H |
| 102 | H | H | H | H | H | CF₃ | H | H | H | H | H | H | H |
| 103 | H | H | H | H | H | CF₃ | H | CH₃ | H | H | H | H | H |
| 104 | H | H | H | H | CF₃ | H | CF₃ | H | H | H | H | H | H |
| 105 | H | F | H | H | H | CN | H | H | H | H | H | H | H |
| 106 | H | F | H | H | H | CF₃ | H | H | H | H | H | H | H |
| 107 | H | F | H | H | H | CF₃ | H | CH₃ | H | H | H | H | H |
| 108 | H | F | H | H | H | CF₃ | H | CH₃ | H | H | H | H | H |
| 109 | H | Cl | H | H | H | CN | H | H | H | H | H | H | H |
| 110 | H | Cl | H | H | H | CF₃ | H | H | H | H | H | H | H |
| 111 | H | Cl | H | H | H | CF₃ | H | CH₃ | H | H | H | H | H |
| 112 | H | Cl | H | H | H | CF₃ | H | CH₃ | H | H | H | H | H |
| 113 | H | Br | H | H | H | CN | H | H | H | H | H | H | H |
| 114 | H | Br | H | H | H | CF₃ | H | H | H | H | H | H | H |
| 115 | H | Br | H | H | H | CF₃ | H | CH₃ | H | H | H | H | H |
| 116 | H | Br | H | H | H | CF₃ | H | CH₃ | H | H | H | H | H |
| 117 | H | H | H | H | H | CN | H | CH₃ | H | H | H | H | H |
| 118 | H | H | H | H | H | Cl | H | H | H | H | H | H | H |
| 119 | H | H | H | H | H | NO₂ | H | Cl | H | H | H | H | H |
| 120 | H | H | H | H | Br | Cl | H | H | H | H | H | H | H |
| 121 | H | F | H | H | H | CN | H | CH₃ | H | H | H | H | H |
| 122 | H | F | H | H | H | Cl | H | H | H | H | H | H | H |
| 123 | H | F | H | H | H | NO₂ | H | Cl | H | H | H | H | H |
| 124 | H | F | H | H | Br | Cl | H | H | H | H | H | H | H |
| 125 | H | Cl | H | H | H | CN | H | CH₃ | H | H | H | H | H |
| 126 | H | Cl | H | H | H | Cl | H | H | H | H | H | H | H |
| 127 | H | Cl | H | H | H | NO₂ | H | Cl | H | H | H | H | H |
| 128 | H | Cl | H | H | Br | Cl | H | H | H | H | H | H | H |
| 129 | H | Br | H | H | H | CN | H | CH₃ | H | H | H | H | H |
| 130 | H | Br | H | H | H | Cl | H | H | H | H | H | H | H |
| 131 | H | Br | H | H | H | NO₂ | H | Cl | H | H | H | H | H |
| 132 | H | Br | H | H | Br | Cl | H | H | H | H | H | H | H |

TABLE 2
Exemplary compounds of Formula (XVIIa):

(XVIIa)

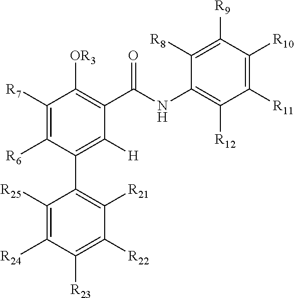

| Compound | R₃ | R₆ | R₇ | R₈ | R₉ | R₁₀ | R₁₁ | R₁₂ | R₂₁ | R₂₂ | R₂₃ | R₂₄ | R₂₅ |
|---|---|---|---|---|---|---|---|---|---|---|---|---|---|
| 201 | H | H | H | H | H | CN | H | H | H | H | H | H | H |
| 202 | H | H | H | H | H | CF₃ | H | H | H | H | H | H | H |
| 203 | H | H | H | H | H | CF₃ | H | CH₃ | H | H | H | H | H |
| 204 | H | H | H | H | CF₃ | H | CF₃ | H | H | H | H | H | H |
| 205 | H | F | H | H | H | CN | H | H | H | H | H | H | H |
| 206 | H | F | H | H | H | CF₃ | H | H | H | H | H | H | H |
| 207 | H | F | H | H | H | CF₃ | H | CH₃ | H | H | H | H | H |
| 208 | H | F | H | H | H | CF₃ | H | CH₃ | H | H | H | H | H |
| 209 | H | Cl | H | H | H | CN | H | H | H | H | H | H | H |
| 210 | H | Cl | H | H | H | CF₃ | H | H | H | H | H | H | H |
| 211 | H | Cl | H | H | H | CF₃ | H | CH₃ | H | H | H | H | H |
| 212 | H | Cl | H | H | H | CF₃ | H | CH₃ | H | H | H | H | H |
| 213 | H | Br | H | H | H | CN | H | H | H | H | H | H | H |
| 214 | H | Br | H | H | H | CF₃ | H | H | H | H | H | H | H |
| 215 | H | Br | H | H | H | CF₃ | H | CH₃ | H | H | H | H | H |
| 216 | H | Br | H | H | H | CF₃ | H | CH₃ | H | H | H | H | H |
| 217 | H | H | H | H | H | CN | H | CH₃ | H | H | H | H | H |
| 218 | H | H | H | H | H | Cl | H | H | H | H | H | H | H |
| 219 | H | H | H | H | H | NO₂ | H | Cl | H | H | H | H | H |
| 220 | H | H | H | H | Br | Cl | H | H | H | H | H | H | H |
| 221 | H | F | H | H | H | CN | H | CH₃ | H | H | H | H | H |
| 222 | H | F | H | H | H | Cl | H | H | H | H | H | H | H |
| 223 | H | F | H | H | H | NO₂ | H | Cl | H | H | H | H | H |
| 224 | H | F | H | H | Br | Cl | H | H | H | H | H | H | H |
| 225 | H | Cl | H | H | H | CN | H | CH₃ | H | H | H | H | H |
| 226 | H | Cl | H | H | H | Cl | H | H | H | H | H | H | H |
| 227 | H | Cl | H | H | H | NO₂ | H | Cl | H | H | H | H | H |
| 228 | H | Cl | H | H | Br | Cl | H | H | H | H | H | H | H |
| 229 | H | Br | H | H | H | CN | H | CH₃ | H | H | H | H | H |
| 230 | H | Br | H | H | H | Cl | H | H | H | H | H | H | H |
| 231 | H | Br | H | H | H | NO₂ | H | Cl | H | H | H | H | H |
| 232 | H | Br | H | H | Br | Cl | H | H | H | H | H | H | H |
| 233 | H | H | H | H | H | CN | H | H | H | F | H | F | H |

In some embodiments of formula (XVIII), the inhibitor has one of formulae (XVIIIb)-(XVIIId):

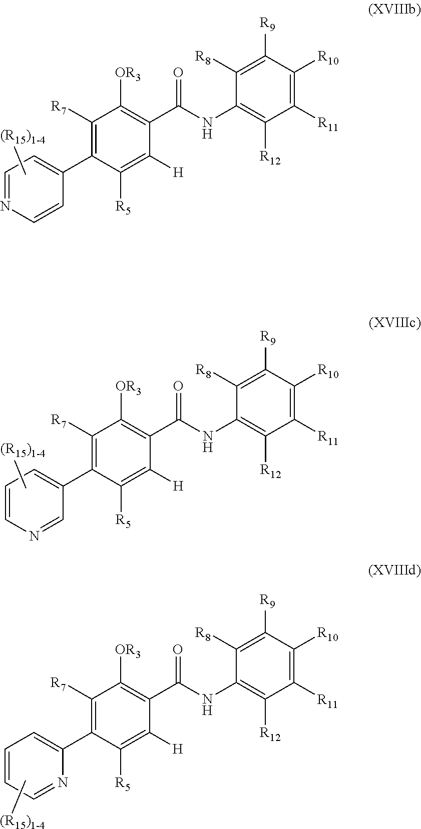

wherein each R₁₅ is independently selected from H, halogen, alkyl, substituted alkyl, alkoxy, substituted alkoxy, hydroxy, cyano, nitro, aryl, substituted aryl, heterocycle, substituted heterocycle, heteroaryl, substituted heteroaryl, amino, substituted amino, carboxy, carboxy ester (e.g., CO₂R where R is alkyl or substituted alkyl), sulfonyl, sulfonate, sulfonamide and substituted sulfonamide. In some instances of formulae (XVIIIb)-(XVIIId), each R₁₅ is independently selected from H, halogen, alkyl, substituted alkyl, cyano and nitro. In certain embodiments of formula (XVIIIb)-(XVIIId), the compound is also a compound of one of formulae (II)-(XII).

In some embodiments of formula (XVIII), the inhibitor has one of formulae (XVIIIe)

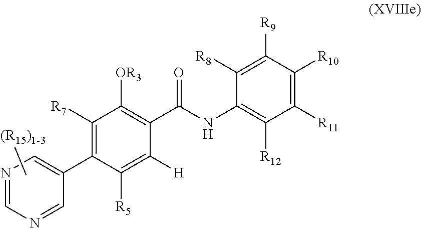

wherein each R₁₅ is independently selected from H, halogen, alkyl, substituted alkyl, alkoxy, substituted alkoxy, hydroxy, cyano, nitro, aryl, substituted aryl, heterocycle, substituted heterocycle, heteroaryl, substituted heteroaryl, amino, substituted amino, carboxy, carboxy ester (e.g., CO₂R where R is alkyl or substituted alkyl), sulfonyl, sulfonate, sulfonamide and substituted sulfonamide. In some instances of formula (XVIIIe), each R₁₅ is independently selected from H, halogen, alkyl, substituted alkyl, cyano and nitro. In certain embodiments of formula (XVIIIe), the compound is also a compound of one of formulae (II)-(XII).

In some embodiments, the inhibitor has one of formulae (XIX)-(XXI):

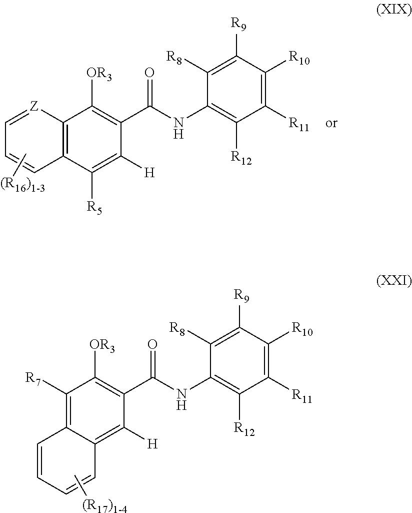

wherein: Z is CR₁₆ or N; and each R₁₆ and each R₁₇ is independently selected from H, halogen, alkyl, substituted alkyl, alkoxy, substituted alkoxy, hydroxy, cyano, nitro, aryl, substituted aryl, heterocycle, substituted heterocycle, heteroaryl, substituted heteroaryl, amino, substituted amino, carboxy, carboxy ester (e.g., CO₂R where R is alkyl or substituted alkyl), sulfonyl, sulfonate, sulfonamide and substituted sulfonamide. In certain embodiments of formula (XIX)-(XXI), the compound is also a compound of one of formulae (II)-(XII).

In some embodiments, the inhibitor is of formula (XIX), wherein: Z is N; and R₁₀ is cyano, trifluoromethyl or halogen. In certain cases, R₁₀ is halogen. In certain cases, R₁₀ is trifluoromethyl. In certain cases, R₁₀ is cyano.

In some embodiments, the inhibitor is of formula (XIX), wherein: Z is N; and R₉ and R₁₀ are independently halogen. In certain cases, R₉ and R₁₀ are selected from fluoro and chloro. In certain cases, R₉ and R₁₀ are fluoro. In certain cases, R₉ and R₁₀ are chloro.

In some embodiments, the inhibitor is of formula (XIX), wherein: Z is N; and R₉ and R₁₁ are independently halogen or trifluoromethyl. In certain cases, R₉ and/or R₁₁ are halogen. In certain cases, R₉ and/or R₁₁ are trifluoromethyl.

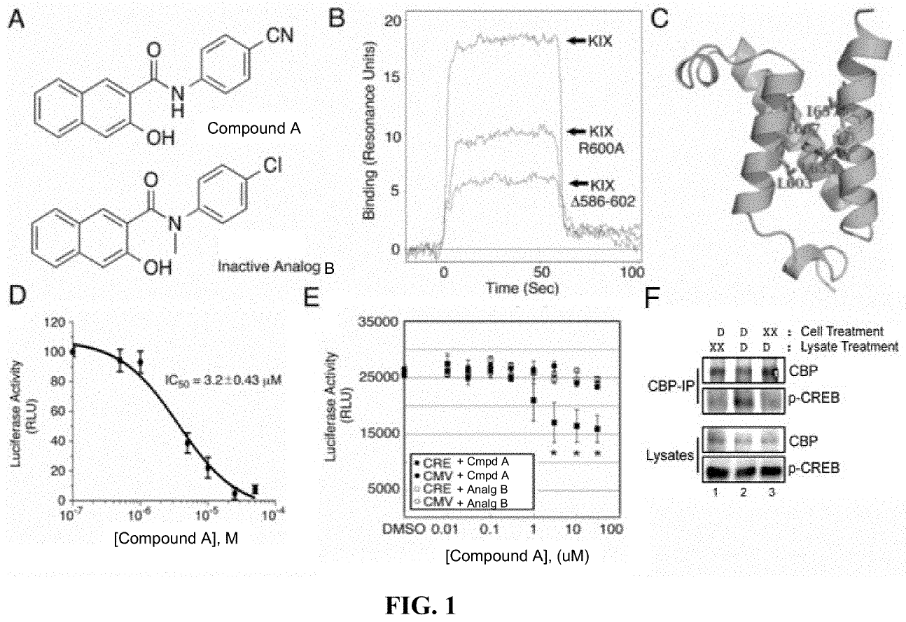

In some embodiments, the inhibitor is of formula (XXI), wherein R₇ is H. In certain instances, R10 is an electron withdrawing group, e.g., cyano. An exemplary compound of formula (XXI) is shown in FIG. 1, panel A, compound A.

In some embodiments of formula (I)-(XVIII), R₅ is H, halogen, aryl, substituted aryl, heteroaryl, substituted heteroaryl, heterocycle (e.g., 2-furanyl) or substituted heterocycle. In some embodiments of formula (I)-(XVIII), R₅ is phenyl or substituted phenyl. In some embodiments of formula (I)-(XVIII), R₅ is pyridyl or substituted pyridyl. In some embodiments of formula (I)-(XVIII), R₅ is 2-furanyl or substituted 2-furanyl.

In certain embodiments, the compound is described by the structure of one of the compounds of Tables 1-3. It is understood that any of the compounds shown in the Tables 1-3 may be present in any convenient salt form. It is understood that prodrug derivatives of any of the compounds shown in Tables 1-3, and any convenient salt forms thereof, are also provided. In some cases, the prodrug derivative is an ester derivative of the salicylamide compound (e.g., R₃ is R′—CO—, where R′ is alkyl or a substituted alkyl). In some cases, the salt form of the compound is a pharmaceutically acceptable salt.

In certain embodiments of formula (I), R₃ and R₅ to R₁₂ are selected from corresponding groups as depicted in any of the compounds of Table 3.

TABLE 3
Exemplary compounds of Formula (I)

(I)

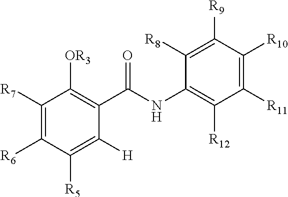

| Com- | | | | | | | | | |
|---|---|---|---|---|---|---|---|---|---|
| pound | R₃ | R₅ | R₆ | R₇ | R₈ | R₉ | R₁₀ | R₁₁ | R₁₂ |
| 1 | H | H | H | H | H | H | CN | H | H |
| 2 | H | Br | H | H | H | H | CN | H | H |
| 3 | H | Cl | H | H | H | H | CN | H | H |
| 4 | H | Cl | H | H | H | Cl | CN | H | H |
| 5 | H | F | H | H | H | Cl | CN | H | H |
| 6 | H | F | H | H | H | Me | CN | H | H |
| 7 | H | Br | H | H | H | Cl | CN | H | H |
| 8 | H | F | H | H | H | H | CN | H | H |
| 9 | H | H | Br | H | H | H | CN | H | H |
| 10 | H | H | F | H | H | H | CN | H | H |
| 11 | H | H | Br | H | Cl | H | CN | H | H |
| 12 | H | H | Br | H | F | H | CN | H | H |
| 13 | H | H | Br | H | H | Cl | CN | H | H |
| 14 | H | H | H | Br | H | H | CN | H | H |
| 15 | H | Ph | H | H | H | H | CN | H | H |
| 16 | H | H | Cl | H | H | H | CN | H | H |
| 17 | H | H | CF₃ | H | H | H | CN | H | H |
| 18 | H | H | CN | H | H | H | CN | H | H |
| 19 | H | F | H | H | H | CN | Me | H | H |
| 20 | H | F | H | H | H | CN | F | H | H |
| 21 | H | F | H | H | H | Me | NO₂ | H | H |
| 22 | H | F | H | H | Cl | H | NO₂ | H | H |
| 23 | H | Cl | H | H | Cl | H | NO₂ | H | H |
| 24 | Ac | Cl | H | H | Cl | H | NO₂ | H | H |
| 25 | H | Cl | H | H | H | Me | NO₂ | H | H |
| 26 | H | F | H | H | H | Cl | Br | H | H |
| 27 | Ac | F | H | H | H | Cl | Br | H | H |
| 28 | C₇H₁₅CO | F | H | H | H | Cl | Br | H | H |
| 29 | 3-methyl- | F | H | H | H | Cl | Br | H | H |
| | butanoyl | | | | | | | | |
| 30 | H | Cl | H | H | H | Cl | Br | H | H |
| 31 | H | F | H | H | H | Cl | Cl | H | H |
| 32 | H | F | H | H | H | F | F | H | H |
| 33 | H | F | H | H | H | F | H | H | H |
| 34 | H | F | H | H | H | Me | Cl | H | H |
| 35 | H | Cl | H | H | H | Me | Cl | H | H |
| 36 | H | F | H | H | Me | H | Cl | H | H |
| 37 | H | F | H | H | F | H | Cl | H | H |
| 38 | H | F | H | H | Cl | H | Cl | H | H |
| 39 | H | F | H | H | F | H | F | H | H |
| 40 | H | F | H | H | F | F | H | H | H |
| 41 | H | F | H | H | H | F | H | F | H |
| 42 | H | F | H | H | H | Cl | H | Cl | H |
| 43 | H | F | H | H | H | Cl | H | H | H |
| 44 | H | F | H | H | H | H | CF₃ | H | H |
| 45 | H | F | H | H | H | H | OCF₃ | H | H |
| 46 | H | F | H | H | H | Cl | OCF₃ | H | H |
| 47 | H | F | H | H | H | CF₃ | Cl | H | H |
| 48 | H | F | H | H | H | CF₃ | F | H | H |
| 49 | H | F | H | H | H | CF₃ | Me | H | H |
| 50 | H | F | H | H | H | CF₃ | H | H | H |
| 51 | H | F | H | H | H | CF₃ | H | CF₃ | H |
| 52 | H | F | H | H | H | OCF₃ | H | H | H |
| 53 | H | F | H | H | H | H | Ph | H | H |
| 54 | H | F | H | H | H | H | OMe | H | H |
| 55 | H | F | H | H | H | OMe | F | H | H |
| 56 | H | F | H | H | H | Me | F | H | H |
| 57 | H | F | H | H | H | H | CO₂Et | H | H |
| 58 | H | F | H | H | H | H | H | CO₂Et | H |
| 59 | H | F | H | H | H | H | H | NMe₂ | H |
| 60 | H | F | H | H | Cl | H | H | H | H |
| 61 | H | F | H | H | F | H | H | H | H |
| 62 | H | F | H | H | CF₃ | H | H | H | H |
| 63 | H | F | H | H | Me | H | H | CF₃ | H |
| 151 | H | Cl | H | H | H | Br | Cl | H | H |
| 152 | H | Cl | H | H | H | H | CF₃ | H | H |
| 153 | H | Cl | H | H | H | Cl | Br | H | CH₃ |
| 154 | H | Cl | H | H | H | H | CF₃ | H | CH₃ |
| 155 | H | Br | H | H | H | Br | Cl | H | H |
| 156 | H | Br | H | H | H | H | CF₃ | H | H |
| 157 | H | Br | H | H | H | Cl | Br | H | CH₃ |
| 158 | H | Br | H | H | H | H | CF₃ | H | CH₃ |
| 159 | H | Br | H | H | H | CF₃ | H | CF₃ | H |
| 160 | H | F | H | H | H | H | NO₂ | H | H |
| 161 | H | Cl | F | H | H | H | CN | H | H |

TABLE 4
Exemplary compounds of Formula (XIX)

| Com- | |
|---|---|
| pound # | Structure |
| 64 | 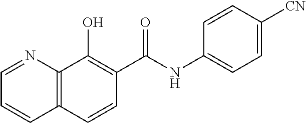 [SMILES: N#Cc1ccc(NC(=O)c2ccc3cccnc3c2O)cc1] |
| 65 | 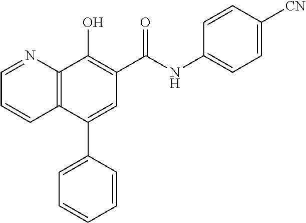 [SMILES: N#Cc1ccc(NC(=O)c2cc(-c3ccccc3)c3cccnc3c2O)cc1] |
| 66 | 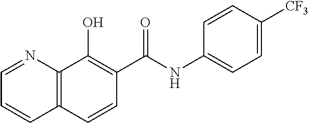 [SMILES: O=C(Nc1ccc(C(F)(F)F)cc1)c1ccc2cccnc2c1O] |
| 67 | 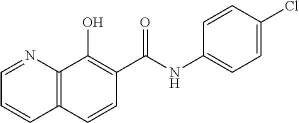 [SMILES: O=C(Nc1ccc(Cl)cc1)c1ccc2cccnc2c1O] |
| 68 | 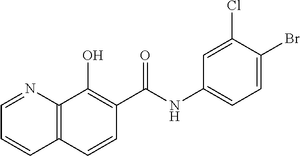 [SMILES: O=C(Nc1ccc(Br)c(Cl)c1)c1ccc2cccnc2c1O] |
| 69 | 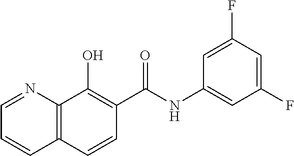 [SMILES: O=C(Nc1cc(F)cc(F)c1)c1ccc2cccnc2c1O] |
| 70 | 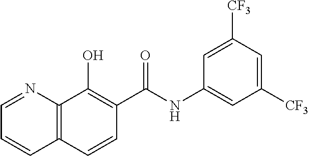 [SMILES: O=C(Nc1cc(C(F)(F)F)cc(C(F)(F)F)c1)c1ccc2cccnc2c1O] |
| 71 | 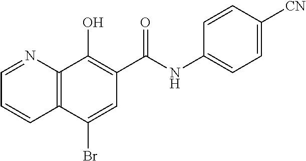 [SMILES: N#Cc1ccc(NC(=O)c2cc(Br)c3cccnc3c2O)cc1] |
| 72 | 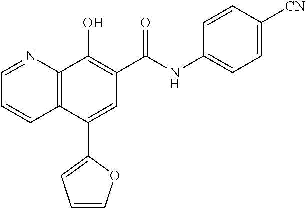 [SMILES: N#Cc1ccc(NC(=O)c2cc(-c3ccco3)c3cccnc3c2O)cc1] |
| 73 | 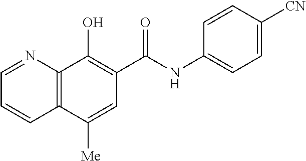 [SMILES: Cc1cc(C(=O)Nc2ccc(C#N)cc2)c(O)c2ncccc12] |
| 74 | 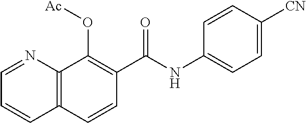 [SMILES: CC(=O)Oc1c(C(=O)Nc2ccc(C#N)cc2)ccc2cccnc12] |
| 75 | 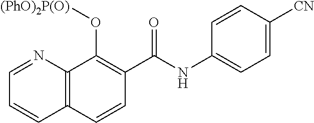 |
| 76 | 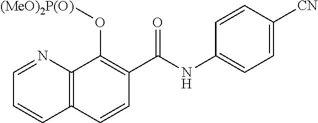 |
| 77 | 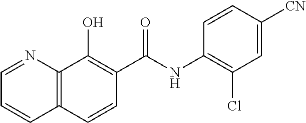 [SMILES: N#Cc1ccc(NC(=O)c2ccc3cccnc3c2O)c(Cl)c1] |
| 78 | 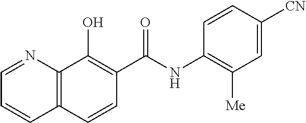 [SMILES: Cc1cc(C#N)ccc1NC(=O)c1ccc2cccnc2c1O] |
| 79 | 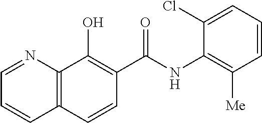 [SMILES: Cc1cccc(Cl)c1NC(=O)c1ccc2cccnc2c1O] |
| 80 | 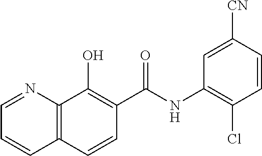 [SMILES: N#Cc1ccc(Cl)c(NC(=O)c2ccc3cccnc3c2O)c1] |
| 81 | 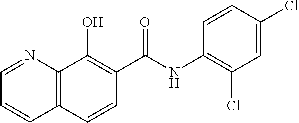 [SMILES: O=C(Nc1ccc(Cl)cc1Cl)c1ccc2cccnc2c1O] |
| 82 | 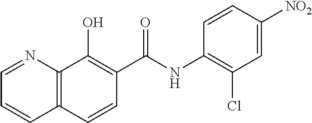 [SMILES: O=C(Nc1ccc([N+](=O)[O-])cc1Cl)c1ccc2cccnc2c1O] |
| 83 | 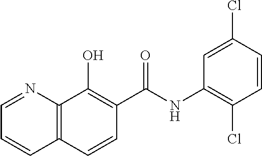 [SMILES: O=C(Nc1cc(Cl)ccc1Cl)c1ccc2cccnc2c1O] |
| 84 | 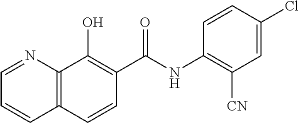 [SMILES: N#Cc1cc(Cl)ccc1NC(=O)c1ccc2cccnc2c1O] |
| 85 | 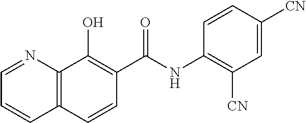 [SMILES: N#Cc1ccc(NC(=O)c2ccc3cccnc3c2O)c(C#N)c1] |
| 86 | 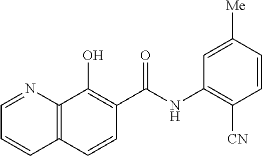 [SMILES: Cc1ccc(C#N)c(NC(=O)c2ccc3cccnc3c2O)c1] |
| 87 | 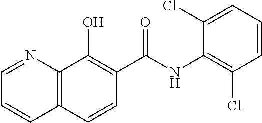 [SMILES: O=C(Nc1c(Cl)cccc1Cl)c1ccc2cccnc2c1O] |
| 88 | 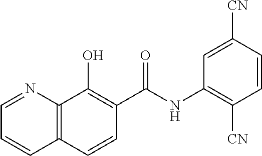 [SMILES: N#Cc1ccc(C#N)c(NC(=O)c2ccc3cccnc3c2O)c1] |
| 89 | 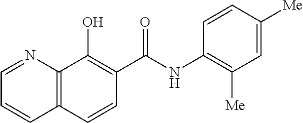 [SMILES: Cc1ccc(NC(=O)c2ccc3cccnc3c2O)c(C)c1] |

Aspects of the present disclosure include CREB transcription inhibitor compounds, salts thereof (e.g., pharmaceutically acceptable salts), and/or solvate, hydrate and/or prodrug forms thereof. In addition, it is understood that, in any compound described herein having one or more chiral centers, if an absolute stereochemistry is not expressly indicated, then each center may independently be of R-configuration or S-configuration or a mixture thereof. It will be appreciated that all permutations of salts, solvates, hydrates, prodrugs and stereoisomers are meant to be encompassed by the present disclosure.

In some embodiments, the subject compounds, or a prodrug form thereof, are provided in the form of pharmaceutically acceptable salts. Compounds containing an amine or nitrogen containing heteraryl group may be basic in nature and accordingly may react with any number of inorganic and organic acids to form pharmaceutically acceptable acid addition salts. Acids commonly employed to form such salts include inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the like salts. In certain specific embodiments, pharmaceutically acceptable acid addition salts include those formed with mineral acids such as hydrochloric acid and hydrobromic acid, and those formed with organic acids such as fumaric acid and maleic acid.

In some embodiments, the subject compounds are provided in a prodrug form. “Prodrug” refers to a derivative of an active agent that requires a transformation within the body to release the active agent. In certain embodiments, the transformation is an enzymatic transformation. Prodrugs are frequently, although not necessarily, pharmacologically inactive until converted to the active agent. “Promoiety” refers to a form of protecting group that, when used to mask a functional group within an active agent, converts the active agent into a prodrug. In some cases, the promoiety will be attached to the drug via bond(s) that are cleaved by enzymatic or non enzymatic means in vivo. Any convenient prodrug forms of the subject compounds can be prepared, e.g., according to the strategies and methods described by Rautio et al. (“Prodrugs: design and clinical applications”, Nature Reviews Drug Discovery 7, 255-270 (February 2008)).

Aspects of the present disclosure include a prodrug form of any one of the compounds described herein, where a promoiety is attached to a hydroxyl group of the compound, e.g., the group designated R₃ is a promoiety (e.g., as described herein). In certain instances of prodrugs of the subject compounds, the promoiety (e.g., attached at R₃) is an acyl or substituted acyl group that forms an ester linkage to the compound. In certain instances of prodrugs of the subject compounds, the promoiety (e.g., attached at R₃) is a phosphate group that forms a phosphate ester linkage to the compound. In certain instances of prodrugs of the subject compounds, the promoiety (e.g., attached at R₃) is an organophosphate group that forms a phosphate triester linkage with the compound. In certain cases, the promoiety has the formula —P(═O)—OR, where R is H, alkyl, substituted alkyl, aryl or substituted aryl. In certain cases, the promoiety has the formula —P(═O)—(OR)₂, where each R is alkyl or substituted alkyl (e.g., —P(═O)—(OMe)₂ or —P(═O)—(OEt)₂). In certain cases, the promoiety has the formula —P(═O)—(OR)₂, where R is aryl or substituted aryl (e.g., —P(═O)—(OPh)₂). In certain instances, the compound is a prodrug derivative of any one of the compounds of formulae (I)-(XXI) and the compounds of Tables 1-4, where the R₃ group is an acyl (e.g., an acetyl), a substituted acyl, or a phosphate ester (e.g., —P(═O)—OR, where R is H, alkyl, substituted alkyl, aryl or substituted aryl).

In some instances, a prodrug form of the subject compound is described by the following [1,3]oxazine-2,4(3H)-dione derivative of formula (I):

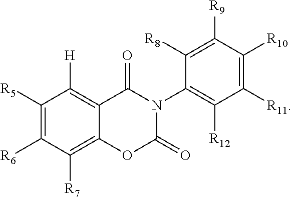

In certain instances, the compound is a [1,3]oxazine-2,4(3H)-dione prodrug derivative of any one of the compounds of formulae (I)-(XXI) and the compounds of Tables 1-4.

In some embodiments, the subject compounds, prodrugs, stereoisomers or salts thereof are provided in the form of a solvate (e.g., a hydrate). The term “solvate” as used herein refers to a complex or aggregate formed by one or more molecules of a solute, e.g. a prodrug or a pharmaceutically-acceptable salt thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

In certain embodiments, the subject compound is modified as a prodrug. Any convenient prodrug modification strategy may be utilized to impart a desired property on the subject compounds, e.g., bioavailability, metabolic half-life, etc. In some cases, the prodrug derivative is an ester derivative of the salicylamide compound, where the phenolic oxygen is derivatized as an ester group. Any convenient ester groups may be utilized, including but not limited to, alkyl, substituted alkyl, aryl, substituted aryl, heterocycle or substituted heterocycle acyl ester groups (e.g., where R₃ of formula (I) is R′—CO—, where R′ is alkyl or a substituted alkyl).

In certain embodiments, the subject compound is modified to include a label, e.g., a fluorescent label, and the subject method further includes detecting the label, if present, in a sample contacted with the compound, e.g., using optical detection.

In certain embodiments, the compound is modified with a support or with affinity groups that bind to a support (e.g. biotin), such that any sample that does not bind to the compound may be removed (e.g., by washing). The specifically bound target protein (e.g., CREB, CBP, or fragment thereof, if present, may then be detected using any convenient means, such as, using the binding of a labeled target specific probe, or using a fluorescent protein reactive reagent. In another embodiment of the subject method, the sample is known to contain the target CREB and CBP.

### Methods

Aspects of the present disclosure include methods of inhibiting a CREB-CBP protein-protein interaction, where a subject inhibitor compound (e.g., as described herein) is brought into contact with a sample including CREB and CBP in an amount and for a period of time sufficient to inhibit the interaction. In some embodiments, the inhibitor compound specifically binds the KIX domain of CREB Binding Protein (CBP), thereby inhibiting interaction of CREB and CBP. The sample can be a cellular sample. The sample can be in vitro or in vivo. The CREB and CBP can be endogenous to any convenient cell of interest. In some instances, the cellular sample includes cells which overexpress CREB. CREB overexpression augments AML cell growth. Inhibition of the CREB-CBP interaction using the subject compounds can lead to disruption of CREB-driven gene expression in a cell of interest.

As such, aspects of the method include contacting a sample with a subject compound (e.g., as described herein) under conditions by which the compound inhibits the CREB-CBP interaction. Any convenient protocol for contacting the compound with the sample may be employed. The particular protocol that is employed may vary, e.g., depending on whether the sample is in vitro or in vivo. For in vitro protocols, contact of the sample with the compound may be achieved using any convenient protocol. In some instances, the sample includes cells that are maintained in a suitable culture medium, and the complex is introduced into the culture medium. For in vivo protocols, any convenient administration protocol may be employed. Depending upon the potency of the compound, the cells of interest, the manner of administration, the number of cells present, various protocols may be employed.

Aspects of the present disclosure include methods for modulating transcription of CREB in a cell that overexpresses CREB. The method can include: contacting the cell with an effective amount of a CREB transcription inhibitor compound to modulate transcription of CREB. By “effective amount” is meant an amount of the compound sufficient to modulate (e.g., increase or decrease by 10% or more, such as 20% or more, 20% or more, 20% or more, 20% or more, 20% or more, 20% or more, 20% or more, 20% or more, or even more) transcription in the cell. In some embodiments, the inhibitor compound specifically binds the KIX domain of CREB Binding Protein (CBP). The inhibitor can have an affinity for the KIX domain of CREB Binding Protein (CBP) that is 1 μM or less, such as 300 nM or less, 100 nM or less, 30 nM or less, 10 nM or less, 3 nM or less, 1 nM or less, or even stronger affinity.

In some embodiments, the subject compounds inhibit CREB-CBP interaction, as determined by an inhibition assay, e.g., by an assay that determines the level of activity related to a CREB function in a cell after treatment with a subject compound, relative to a control, by measuring the IC₅₀ or EC₅₀ value, respectively. In certain embodiments, the subject compounds have an IC₅₀ value (or EC₅₀ value) of 10 μM or less, such as 3 μM or less, 1 μM or less, 500 nM or less, 300 nM or less, 200 nM or less, 100 nM or less, 50 nM or less, 30 nM or less, 10 nM or less, 5 nM or less, 3 nM or less, 1 nM or less, or even lower. In certain assays, a subject compound may inhibit its target with an IC₅₀ of 1×10⁻⁶ μM or less (e.g., 1×10⁻⁶ μM or less, 1×10⁻⁷ μM or less, 1×10⁻⁸ μM or less, 1×10⁻⁹ μM or less, 1×10⁻¹⁰ μM or less, or 1×10⁻¹¹ μM or less).

In certain embodiments, the subject compounds have no significant effect on the viability of a mammalian cell, as determined by a cell cytotoxicity assay, e.g., as determined by administering a subject compound to a HeLa cell and determining the number of viable cells present. The subject compounds may exhibit a % cell viability, as compared to a control (e.g., a DMSO control), of 15% or more, such as 20% or more, 30% or more, 40% or more, 50% or more, 60% or more, 70% or more, 80% or more, 90% or more, 100% or more, 120% or more, or even higher. The subject compounds may exhibit a CC₅₀ value of 1 nM or higher, such as 100 nM or higher, 300 nM or higher, 1 μM or higher, 3 μM or higher, 5 μM or higher, 10 μM or higher, 20 μM or higher, 30 μM or higher, 50 μM or higher, or even higher.

In certain embodiments, the compounds have a therapeutic index (e.g., the ratio of a compound's cytotoxicity (e.g., cell cytotoxicity, CC50) to bioactivity (e.g., inhibition activity, IC50)) that is 20 or more, such as 50 or more, 100 or more, 200 or more, 300 or more, 400 or more, 500 or more, or even more.

The protocols that may be employed in determining the subject inhibition activity are numerous, and include but are not limited to cell-free assays, e.g., binding assays; assays using purified CREB and/or CBP, or fragments thereof, cellular assays in which a cellular phenotype is measured, e.g., gene expression assays; and in vivo assays that involve a particular animal (which, in certain embodiments may be an animal model for a condition related to the target indication).

In some embodiments, the subject method is an in vitro method that includes contacting a sample with a subject compound that specifically inhibits a CREB-CBP protein-protein interaction. In certain instances of the method, the compound that is used to contact the sample is a compound of one of formulae (I)-(XXI). In certain instances of the method, the compound that contacts the sample is described by one of the compounds of Tables 1-4.

Aspects of the present disclosure include methods of inhibiting proliferation of a cancer cell. The method may include contacting the cell with a subject compound (e.g., as described herein). In some embodiments, the method is a method of inhibiting proliferation of cancer cells by inhibiting a CREB-CBP interaction of a cancer cell that overexpresses CREB. In addition, by utilizing such a target, the methods of the disclosure allow targeting of the cancer cell without having an adverse effect on normal cells, thereby substantially eliminating toxicity induced side effects. The methods also provide anti-cancer therapies for a variety of cancers, including hematologic malignancies. In some instances, the hematologic malignancy is a leukemia, such as AML or ALL. In certain instances of the method, the compound that is used to contact the cell is a compound of one of formulae (I)-(XXI). In certain instances of the method, the compound that contacts the cell is described by one of the compounds of Tables 1-4.

In some embodiments, the subject method is a method of treating a subject for cancer, including a hematologic malignancy or leukemia such as AML or ALL. In some embodiments, the subject method includes administering to the subject an effective amount of a subject compound (e.g., as described herein) or a pharmaceutically acceptable salt thereof. The subject compound may be administered as part of a pharmaceutical composition (e.g., as described herein). In certain instances of the method, the compound that is administered is a compound of one of formulae (I)-(XXI). In certain instances of the method, the compound that is administered is described by one of the compounds of Tables 1-4.

In some embodiments, an “effective amount” is an amount of a subject compound that, when administered to an individual in one or more doses, in monotherapy or in combination therapy, is effective to ameliorate at least one symptom in the individual by at least about 20% (20% amelioration), at least about 30% (30% amelioration), at least about 40% (40% amelioration), at least about 50% (50% amelioration), at least about 60% (60% amelioration), at least about 70% (70% amelioration), at least about 80% (80% amelioration), or at least about 90% (90% amelioration), compared to an individual in the absence of treatment with the compound, or alternatively, compared to the individual before or after treatment with the compound.

In some embodiments, an “effective amount” of a compound is an amount that, when administered in one or more doses to an individual in need thereof, is effective to achieve a 1.5-log, a 2-log, a 2.5-log, a 3-log, a 3.5-log, a 4-log, a 4.5-log, or a 5-log reduction in cancer cells in a sample of the individual.

In some embodiments, an effective amount of a compound is an amount that ranges from about 50 ng/ml to about 50 μg/ml (e.g., from about 50 ng/ml to about 40 μg/ml, from about 30 ng/ml to about 20 μg/ml, from about 50 ng/ml to about 10 μg/ml, from about 50 ng/ml to about 1 μg/ml, from about 50 ng/ml to about 800 ng/ml, from about 50 ng/ml to about 700 ng/ml, from about 50 ng/ml to about 600 ng/ml, from about 50 ng/ml to about 500 ng/ml, from about 50 ng/ml to about 400 ng/ml, from about 60 ng/ml to about 400 ng/ml, from about 70 ng/ml to about 300 ng/ml, from about 60 ng/ml to about 100 ng/ml, from about 65 ng/ml to about 85 ng/ml, from about 70 ng/ml to about 90 ng/ml, from about 200 ng/ml to about 900 ng/ml, from about 200 ng/ml to about 800 ng/ml, from about 200 ng/ml to about 700 ng/ml, from about 200 ng/ml to about 600 ng/ml, from about 200 ng/ml to about 500 ng/ml, from about 200 ng/ml to about 400 ng/ml, or from about 200 ng/ml to about 300 ng/ml).

In some embodiments, an effective amount of a compound is an amount that ranges from about 10 pg to about 100 mg, e.g., from about 10 pg to about 50 pg, from about 50 pg to about 150 pg, from about 150 pg to about 250 pg, from about 250 pg to about 500 pg, from about 500 pg to about 750 pg, from about 750 pg to about 1 ng, from about 1 ng to about 10 ng, from about 10 ng to about 50 ng, from about 50 ng to about 150 ng, from about 150 ng to about 250 ng, from about 250 ng to about 500 ng, from about 500 ng to about 750 ng, from about 750 ng to about 1 μg, from about 1 μg to about 10 μg, from about 10 μg to about 50 μg, from about 50 μg to about 150 μg, from about 150 μg to about 250 μg, from about 250 μg to about 500 μg, from about 500 μg to about 750 μg, from about 750 μg to about 1 mg, from about 1 mg to about 50 mg, from about 1 mg to about 100 mg, or from about 50 mg to about 100 mg. The amount can be a single dose amount or can be a total daily amount. The total daily amount can range from 10 pg to 100 mg, or can range from 100 mg to about 500 mg, or can range from 500 mg to about 1000 mg.

In some embodiments, a single dose of a compound is administered. In other embodiments, multiple doses are administered. Where multiple doses are administered over a period of time, the compound can be administered twice daily (qid), daily (qd), every other day (qod), every third day, three times per week (tiw), or twice per week (biw) over a period of time. For example, a compound is administered qid, qd, qod, tiw, or biw over a period of from one day to about 2 years or more. For example, a compound is administered at any of the aforementioned frequencies for one week, two weeks, one month, two months, six months, one year, or two years, or more, depending on various factors.

Administration of an effective amount of a subject compound to an individual in need thereof can result in one or more of: 1) a reduction in cancer cells in a target biological sample; 2) a reduction in the spread of cancer cells in an individual; 3) an increase in the rate of sustained response to therapy; 4) a reduction of morbidity or mortality in clinical outcomes; 5) shortening the total length of treatment when combined with other chemotherapeutic agents; and 6) an improvement in an indicator of disease response (e.g., a reduction or amelioration in one or more symptoms). Any of a variety of methods can be used to determine whether a treatment method is effective. For example, a biological sample obtained from an individual who has been treated with a subject method can be assayed. In certain cases, the cancer cells are hematologic cancer cells.

In some embodiments, the subject is human. The subject may be in need of treatment for a cancer. In some instances, the subject methods include diagnosing a cancer, including any one of the cancer indications described herein. In some embodiments, the compound is administered as a pharmaceutical preparation.

In certain embodiments, the compound is a modified compound that includes a label, and the method further includes detecting the label in the subject. The selection of the label depends on the means of detection. Any convenient labeling and detection systems may be used in the subject methods, see e.g., Baker, “The whole picture,” Nature, 463, 2010, p 977-980. In certain embodiments, the compound includes a fluorescent label suitable for optical detection. In certain embodiments, the compound includes a radiolabel for detection using positron emission tomography (PET) or single photon emission computed tomography (SPECT). In some cases, the compound includes a paramagnetic label suitable for tomographic detection. The subject compound may be labeled, as described above, although in some methods, the compound is unlabelled and a secondary labeling agent is used for imaging.

### Pharmaceutical Compositions

The herein-discussed compounds can be formulated using any convenient excipients, reagents and methods. Compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the subject compound is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures. In some embodiments, the subject compound is formulated for sustained release.

In another aspect of the present invention, a pharmaceutical composition is provided, comprising, or consisting essentially of, a compound of the present invention, or a pharmaceutically acceptable salt, isomer, tautomer or prodrug thereof, and further comprising one or more additional agents of interest. In some embodiments, the subject compound and a chemotherapeutic agent, are administered to individuals in a formulation (e.g., in the same or in separate formulations) with a pharmaceutically acceptable excipient(s). Any convenient agents can be utilized in the subject methods in conjunction with the subject compounds. The subject compound and second agent, as well as additional therapeutic agents as described herein for combination therapies, can be administered orally, subcutaneously, intramuscularly, parenterally, or other route. The subject compound and second agent may be administered by the same route of administration or by different routes of administration. The therapeutic agents can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ.

In some embodiments, the subject compound and a second agent of interest are administered to individuals in a formulation (e.g., in the same or in separate formulations) with a pharmaceutically acceptable excipient(s). Second active agents of interest include anticancer agents, including but not limited to, nucleoside and nucleotide analog chemotherapeutic drugs, such as cytarabine and anthracycline family drugs such as daunorubicin, doxorubicin, epirubicin and idarubicin. The subject compound and second agent, as well as additional therapeutic agents as described herein for combination therapies, can be administered orally, subcutaneously, intramuscularly, parenterally, or other route. The subject compound and second agent may be administered by the same route of administration or by different routes of administration. The therapeutic agents can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ.

The subject compounds may be administered in a unit dosage form and may be prepared by any methods well known in the art. Such methods include combining the subject compound with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and so e.g., chloroquine, primaquine, mefloquine, doxycycline, atovaquone-proguanil, quinine, quinidine, artesunate, artemether, lumefantrine; etc. lution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

Any drug delivery device or system that provides for the dosing regimen of the instant disclosure can be used. A wide variety of delivery devices and systems are known to those skilled in the art.

### Utility

The compounds and methods of the invention, e.g., as described herein, find use in a variety of applications. Applications of interest include, but are not limited to: research applications and therapeutic applications. Methods of the invention find use in a variety of different applications including any convenient application where inhibition of a CREB-CBP protein-protein interaction is desired.

The subject compounds and methods find use in a variety of research applications. The subject compounds and methods may be used in elucidating a mechanism involving CREB transcription or CREB-CBP binding. The subject compounds and methods may be used in the optimization of the bioavailability and metabolic stability of compounds.

The subject compounds and methods find use in a variety of therapeutic applications. Therapeutic applications of interest include any indications in which overexpression of CREB is implicated as a cause or a compounding factor in disease progression. As such, the subject compounds find use in the treatment of a variety of different cancers. For example, the subject compounds and methods may find use in treating a hematologic malignancy such as AML or ALL.

### EXAMPLES

The following examples are put forth so as to provide those of ordinary skill in the art with a complete disclosure and description of how to make and use embodiments of the present disclosure, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present disclosure. All such modifications are intended to be within the scope of the claims appended hereto.

### Example 1: General Synthetic Procedure

A dry, 50 mL round bottom flask was charged with a magnetic stir bar then sealed with a septum and flushed with nitrogen. To this was added the salicylic acid derivative (2.0 mmol, 1.0 equiv) and dichloromethane (10 mL). While stirring, SOCl₂ (730 μL, 10 mmol, 5.0 equiv) was added dropwise, followed by dimethylformamide (4 μL, 0.05 mmol, 0.03 equiv), and the mixture was allowed to stir at room temperature under nitrogen for 12 hours. At this point, the mixture was concentrated under vacuum to afford the solid acid chloride derivative which was used immediately without further purification. The flask containing the acid chloride was then re-sealed with a septum and placed under a nitrogen atmosphere, and its contents were re-suspended in fresh dichloromethane (20 mL). While stirring, the aniline derivative (4.0 mmol, 2.0 equiv) was added and the resulting opaque mixture was stirred for an additional 18 h. At this point, the mixture was quenched with ice water (5 mL) and phases were separated using a separatory funnel. The aqueous layer was extracted twice with dichloromethane (20 mL) then the organic layers were combined and washed with brine (40 mL). The organic layer was then dried over Na₂SO₄, filtered, and concentrated under vacuum to afford a crude solid residue. This residue was then re-suspended in dichloromethane (10 mL) and adsorbed onto silica gel (1 g), then chromatographed on a 12 μg silica gel column using a solvent gradient of 0-40% ethyl acetate in hexanes over 25 min. The product-containing fractions were combined then concentrated under vacuum to afford the purified salicylamide derivative.

This general procedure was adapted to prepare a variety of compounds of Tables 1-4.

### Synthesis of N-(4-Cyanophenyl)-3-Hydroxy-2-naphthamide (Compound A)

Thionyl chloride (20 mL) was added to 3-hydroxy-2-naphthoic acid (8.5 g, 45 mmol) at room temperature. The resulting mixture was then heated under reflux for 1 hour. Excess thionyl chloride was removed under reduced pressure and the residue was dissolved with THF (80 mL). 4-Chloroaniline (8.0 g, 67.5 mmol) was then added to this solution and the mixture was heated under reflux for another 1 hour. The reaction mixture was cooled to room temperature, diluted with 1 N HCl (40 mL) and stirred at room temperature for 30 min. The precipitate was collected by filtration and washed with dichloromethane to yield Compound A (10.2 g, 79%) as an off-white solid: m.p. 267-268° C. ¹H NMR (400 MHz, DMSO-d₆) δ 11.08 (s, 1H), 10.88 (s, 1H), 8.41 (s, 1H), 7.98 (d, J=8.5 Hz, 2H), 7.95 (d, J=8.1 Hz, 1H), 7.86 (d, J=8.9 Hz, 2H), 7.78 (d, J=8.3 Hz, 1H), 7.52 (t, J=7.6 Hz, 1H), 7.37 (t, J=7.6 Hz, 1H), 7.34 (s, 1H); ¹³C NMR (100 MHz, DMSO-d₆) δ 153.01, 142.94, 135.71, 133.30, 130.66, 128.68, 128.16, 126.89, 125.81, 123.81, 122.97, 120.14, 119.04, 110.43, 105.52. The purity of Compound A was measured by RP-HPLC and was found to be >99%.

### Example 2: Activity of Assays

### Protein Purification and Biacore

KIX domain mutants were created by standard cloning and mutagenesis methods in the pGEX4T3 vector (GE Healthcare Life Sciences). GST-KIX fusion proteins were expressed in BL21(DE3-) cells (New England Biolabs) following induction with 1 mM IPTG for 6 hours at 37 degrees. GST-KIX and its mutants were purified with the B-PER GST Fusion Protein Spin Purification Kit (Thermo Scientific/Pierce). Surface Plasmon Resonance (Biacore) analysis was performed on a GE Biacore 3000 surface plasmon resonance instrument in collaboration with the Stanford Protein and Nucleic Acid (PAN) Facility.

### AML Cell Lines and Patient Samples

AML cell lines were purchased from ATCC and maintained with IMDM (Gibco) supplemented with 10% FBS (Fisher Scientific) and 1% PSG (Gibco). Cells were plated at a density of 2-4×10⁵ cells/ml, and treated with various doses of Compound A in 0.1% DMSO or 0.1% DMSO alone. Cell counts and viability were determined using the Vi-CELL XR Cell Viability Analyzer (Beckman Coulter). HL-60 and KG-1 cells overexpressing CREB or CBP were generated using lentiviral gene delivery with subsequent puromycin selection and FACs sorting for GFP. CREB knockdown was achieved by infecting cells with a lentivirus expressing the shRNA sequence 5′-GCAAATGACAGTTCAAGCCC-3′ (SEQ ID NO:). For chemotherapy combination experiments, combination index values were calculated using median effects analysis on Calcusyn software. Human patient bone marrow samples were cultured in DMEM plus 20% FBS and 1×PSG, supplemented with recombinant GM-CSF (20 ng/ml), G-CSF (20 ng/ml), SCF (50 ng/ml), IL-3 (20 ng/ml), and IL-6 (10 ng/ml). Cells were plated at a concentration of 1×10⁵ cells/ml in a 12-well plate. Vehicle (0.1% DMSO) or Compound A (2 μM) was added for up to 72 hours. All samples contained >85% AML blasts and were not sorted prior to performing experiments. Immunostaining and flow cytometry analyses were performed according to standard procedures. All antibodies were purchased from BD Biosciences. Single cell suspensions of bone marrow culture were subsequently analyzed on a D×P10 flow cytometer (Cytek). Bone marrow from AML patients were collected through voluntary patient participation at University of California, Los Angeles (Los Angeles, Calif., USA) and Stanford University (Palo Alto, Calif., USA) in compliance with the Institutional Review Board regulations of each institution.

### Luciferase Assays

KG-1 cell lines were created to express luciferase in a CREB-dependent or non-CREB-dependent fashion using lentiviral gene delivery. Cells were sorted for mCherry expression by flow cytometry and selected with puromycin. Luciferase activity was measured on a spectrophotometer using the Promega Luciferase Activity Kit (Promega) per manufacturer's instructions following six hours of treatment with Compound A or 0.1% DMSO. The split Renilla luciferase complementation assay has been described previously (Li BX, and Xiao X. Discovery of a small-molecule inhibitor of the KIX-KID interaction. Chembiochem: a European journal of chemical biology. 2009; 10(17):2721-4). In this assay, the KID and KIX domains were fused to the N- and C-terminal regions of Renilla luciferase, respectively. Once KIX binds phosphorylated KID, the Renilla luciferase regions were brought together, resulting in luciferase activity.

### Cell Cycle Analysis

KG-1 cells were synchronized at prometaphase using a modified thymidine plus nocodazole block (Whitfield et al. identification of genes periodically expressed in the human cell cycle and their expression in tumors. Mol Biol Cell. 2002; 13(6):1977-2000). Briefly, KG-1 cells were treated with 2 mM thymidine for 30 h, washed with PBS and released from G₁/S block in fresh media for 4 h. The cells were incubated with 300 nM nocodazole (Sigma) for 13 h. Compound A or DMSO was added 3 hours before release. The synchronized cells were washed with PBS and released from the mitotic block in fresh media containing Compound A or DMSO. To analyze DNA content by flow cytometry, cells were harvested, fixed in 70% ice-cold ethanol for at least 1 hour at −20° C., and then incubated in propidium iodide (PI) staining buffer (PBS containing RNase A (50 μg/ml), 0.1% sodium citrate, and PI (50 sg/ml)) for 30 minutes at RT. Cells were analyzed on a FACS Calibur flow cytoneter (BD Biosciences). Cell-cycle distribution was determined using FlowJo software (TreeStar).

### Chromatin Immunoprecipitation and High-Throughput Sequencing (RNA-Seq and ChIP-Seq)

For Chip-Seq experiments, KG-1 cells were treated with 5 μM Compound A or DMSO for 6 hours. Cells were cross-linked with 1% formaldehyde at room temperature for 10 min and then incubated with 0.125 mM glycine for 5 min. After cross-linking, chromatin was digested by Micrococcal nuclease and then sonicated using SimpleChIP® Plus Enzymatic Chromatin IP Kit (Cell Signaling, Danvers, Mass.) following the manufacturer's protocol. Chromatin immunoprecipitations were carried out with anti-CREB antibody (Millipore, Billerica, Mass.) or a control IgG (Santa Cruz Biotechnology). The captured immunocomplexes containing bound transcriptional DNA fragments were eluted, with recovered DNA fragments used for PCR amplification. For RNA-Seq experiments, KG-1 cells were treated with Compound A (5 μM) or 0.1% DMSO for 12 hours. RNA was prepared using the Aurum Total RNA Mini Kit (Bio-Rad). Sample libraries were run using the Illumina sequencing platform. Two hundred million reads were collected on two biological replicates of the experiment. Libraries were prepared using the Illumina Truseq RNA samples prep kit per manufacturer's instructions. Fastq files were aligned using TopHat. Aligned BAM files were used for CuffDiff calculation of differentially expressed genes. CummeRbund R package was used to infer QC of the RNA-seq (GEO Submission GSE74928) [NCBI tracking system #17594369]. ChIP-seq analysis was performed. BETA analysis was used by inferring differentially expressed genes between DMSO and Compound A treated cells based on a fdr≤0.01 and gene distance of 100 kb (Galaxy/cistrome). DNA sequences enriched on ChIP-Seq were defined.

### RT-PCR

Total RNA was isolated using the Aurum RNA Isolation Mini Kit, and the iScript cDNA Synthesis Kit was used to prepare samples for qPCR (Bio-Rad). PCR was performed on the CFX384 Real-Time System (Bio-Rad) and results were analyzed using the Livak method. For analysis of RNA-seq data as target genes of known transcription factors, a curated list of all human target genes was extracted from the TRANSFAC Pro database.

### Western Blot Analysis

Cells were lysed in RIPA buffer (Sigma), and the protein concentrations were determined by BCA assay (Thermo Scientific Pierce). Lysates were solubilized in 6×SDS PAGE buffer and 40-80 mg of protein was loaded into 6-15% SDS PAGE gels, pheresed, and then transferred onto 0.2-0.45 mm PVDF membrane (Millipore). Primary antibodies were applied at a dilution of 1:1000. Anti-Bcl-2, -Bax, -Mcl-1, -Bcl-XL, -Cyclin A and -Cyclin D1 antibodies were purchased from Cell Signaling Technologies. Anti-total acetylated histone and -ac-H3K27 were purchased from Santa Cruz Biotechnologies. Anti-actin and -CREB antibodies were purchased from upstate. Secondary antibodies were used at a 1:2500 dilution and purchased from Thermo Scientific/Pierce. WesternBright ECL was used for image acquisition on Image Lab software (Bio-Rad).

### Hematopoietic Cell Colony Assays

Human bone marrow cells from non-leukemic patients were resuspended in a volume of 0.3 mL of IMDM with 20% FBS at 5×10⁵ cells/mL and mixed with varying concentrations of the Compound A (10 pM to 10 mM in 0.1% DMSO) or 0.1% DMSO control. This was added to 3 mL of methylcellulose-containing growth factors IL-3, IL-6, G-CSF, erythropoietin, SCF (Methocult GF-H4434; Stem Cell Technology) and plated. The colonies were observed daily and counted on day 14.

### Caspase-3 Activity

The ApoTarget Caspase-3 Protease Activity Kit (Invitrogen) was used per manufacturer's instructions.

### Multiparameter Single Cell Mass Cytometry (CyTOF)

Bone marrow from primary AML patients were cultured as above and treated for 48 hours with 2 μM Compound A or 0.1% DMSO control. These were stained for viability using cisplatin. Cells were fixed with 1.6% PFA (Electron Microscopy Sciences) for 10 min and washed with cell staining media (CSM). Fc receptor block was performed using Human TruStain FcX (Biolegend) following manufacturer's instructions. Cells were stained for surface proteins at room temperature for 30 min. Following staining, cells were washed twice with CSM and permeabilized with methanol pre-cooled to 4° C. for 10 min. Cells were then washed twice and stained for intracellular proteins for 30 min at room temperature. Surface and intracellular staining cocktails are listed in supplementary methods. Cells were washed and stained with 1 mL of 2000× iridium DNA intercalator (diluted 1:5000 in PBS with 1.6% PFA; DVS Sciences) overnight at 4° C. Data were acquired using internal metal isotope bead standards as previously described (Finck et al., “Normalization of mass cytometry data with bead standards.” Cytometry A. 2013; 83(5):483-94). Cell events were acquired at approximately 500 events per second on a CyTOF I (DVS Sciences/Fluidigm). All antibodies were also purchased from DVS Sciences/Fluidigm. Each patient sample was individually normalized to the internal bead standards prior to analysis. To remove dead cells and debris, cells were gated based on cell length and DNA content as described (Bendall et al., Single-cell mass cytometry of differential immune and drug responses across a human hematopoietic continuum. Science. 2011; 332(6030):687-9).

### Statistical Analysis

Unless noted, all experiments were performed in triplicate, and Student's t-test was used to assess experimental mean values for statistically significant differences, with p-values of <0.05 deemed statistically significant.

### AML Xenograft Models of AML

For AML xenograft mouse experiments, HL-60 cells (2×10⁶) expressing firefly luciferase and GFP, or cryopreserved human AML patient cells (5×10⁶), were injected through the tail vein into NOD.Cg-prkdc^{scid} Il2rg^{tm1Wjl}SzJ (NSG) mice. Mice were then Compound A or 10% DMSO, injected intravenously by tail vein daily, until death or an endpoint was reached (moribundity) in accordance with the animal care institutional guidelines. All mouse experiments were subject to institutional approval by Stanford University Institutional Animal Care and Use Committee. Leukemia progression in mice at the indicated time points was monitored using an in vivo IVIS 100 bioluminescenceoptical imaging system (Xenogen Corporation). D-Luciferin (Promega) dissolved in sterile phosphate-buffered saline was injected intraperitoneally at a dose of 2.5 mg/kg, 15 minutes before measuring the luminescent signal. General anesthesia was induced with 2 isoflurane and continued during the procedure using a nose cone. Analysis was performed on Living Image In Vivo imaging software (Perkin-Elmer). Imaging was performed in collaboration with the Stanford Small Animal Imaging Facility. Bone marrow was aspirated from bone marrow cavities, and GFP+ cells were sorted using the FACSCalibur flow cytometer (BD Biosciences).

### Results and Discussion

### Compound A is a Competitive Inhibitor of CREB Binding to CBP

The region of CBP critical for binding Ser-133-phosphorylated-CREB is termed the Kinase-Inducible Acceptor domain or ‘KIX’ domain, and spans CBP amino acids 586-666. Multidimensional NMR data demonstrated that the CREB docking surface of this domain is comprised of several hydrophobic residues found in alpha helices al and a3, which form a shallow binding groove (Radhakrishnan et al. Solution structure of the KIX domain of CBP bound to the transactivation domain of CREB: a model for activator:coactivator interactions. Cell. 1997; 91(6):741-52). A molecule was tested for targeting of this domain and disruption of the CREB-CBP interaction, termed “Compound A” (N-(4-cyanophenyl)-3-hydroxy-2-naphthamide) (FIG. 1, panel A). Structure-activity relationship studies reveal Analog B as an inactive yet similarly structured compound (N-methyl-(4-chlorophenyl)-3-hydroxy-naphthamide).

To confirm the binding of Compound A to the KIX domain of CBP, the KIX domain was expressed and purified as a fusion protein with Glutathione-S-Transferase (GST). Binding of Compound A to the KIX domain and two KIX domain mutants in which residues critical for CREB binding were altered or removed (Arg-600 mutated to Alanine or deletion of amino acids 586-602) was determined by Surface Plasmon Resonance (Biacore) analysis (FIG. 1, panel B). Mutation of Arginine-600 to Alanine reduced binding of Compound A by ˜45%, while a KIX domain mutant lacking amino acids 586-602 reduced binding by ˜70%. These data confirm that binding of Compound A to CBP is mediated by the same amino acids responsible for binding CREB, positioning Compound A as a competitive inhibitor of CREB binding to CBP. A hypothetical binding model between Compound A and CBP KIX domain is shown (FIG. 1, panel C). This binding model suggests the major interactions between Compound A and the KIX domain are hydrophobic. The aniline ring of Compound A is predicted to project into a hydrophobic pocket of KIX where Leucine-141 of CREB, an amino acid essential for stable CREB binding, normally docks.

To test the ability of Compound A to disrupt CREB-CBP binding in cells, a split Renilla luciferase complementation assay was performed. Compound A inhibited the interaction between CREB and CBP with an IC₅₀ of 3.20±0.43 μM in 293T cells treated with forskolin (FIG. 1, panel D). To determine whether Compound A could specifically decrease CREB-driven reporter gene expression in AML cells, KG-1 AML cell lines expressing luciferase under the control of a CREB-driven promoter (two CRE elements placed within −200 of the ATG start site, 5′ to an attenuated CMV promoter) or a non-CRE-element-containing, CMV-driven promoter, were generated and treated with Compound A. CREB-driven luciferase activity was reduced by Compound A in a dose-dependent fashion after 6 hours of treatment, whereas CMV-driven luciferase expression was unchanged following treatment (FIG. 1, panel E). This early timepoint was selected to permit accurate reporter gene activity measurement prior to the onset of anticipated global cell dysfunction or death induced by Compound A. Treatment with 3 μM Compound A reduced luciferase activity by 33.2±7.7% at this timepoint. The inactive analog Analog B had no significant effect on luciferase activity in either AML cell line. Finally, it was demonstrated that treatment of HEK293 lysates or cells with Compound A prevented binding of CREB to CBP (FIG. 1, panel F). Thus, Compound A inhibits CREB function through disruption of CREB:CBP interaction.

### CREB Expression Levels Determine Compound A Potency and Efficacy

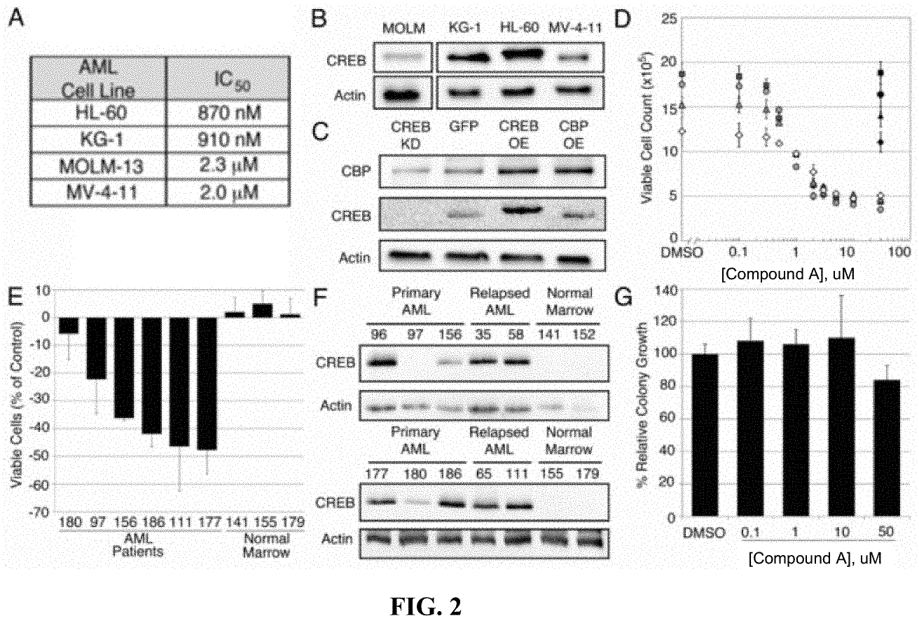

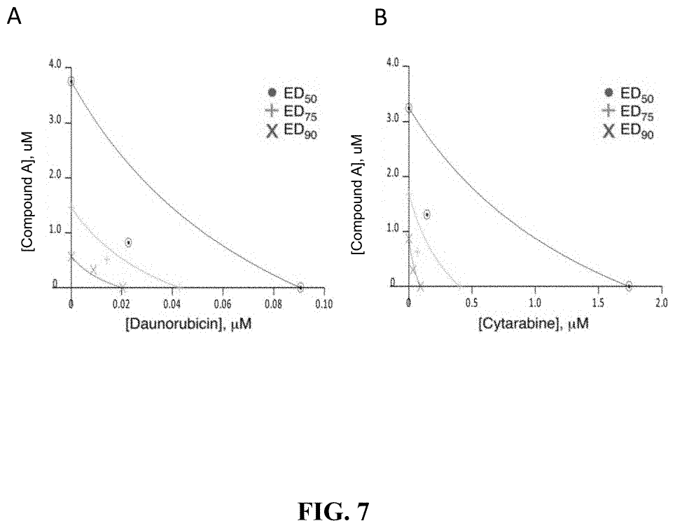

To test the effects of CREB inhibition on AML cells, four AML cell lines were treated with doses of Compound A ranging from 100 pM to 10 μM for 48 hours. The IC₅₀ of Compound A for these AML cell lines, defined as a 50% reduced viable cell count compared to DMSO-treated cells after 48 hours of culture, ranged from 870 nM to 2.3 μM (FIG. 2A). Western blot analysis showed that in these four cell lines, higher CREB expression was associated with increased Compound A potency, as evidenced by a lower IC₅₀ value (FIG. 2B). KG-1 cells were less sensitive to Compound A and expressed lower levels of CREB compared to HL-60 cells (FIG. 2, panel A). CREB was overexpressed to determine whether sensitivity of KG-1 cells to Compound A could be increased. KG-1 cell lines in which CREB or CBP was overexpressed, or CREB was knocked down by shRNA, were generated (FIG. 2C). A KG-1 cell line expressing only GFP was used as a control. These cell lines were treated with a range of Compound A concentrations for 48 hours (FIG. 2D). Experiments were also performed to examine the efficacy of combining Compound A with cytarabine or daunorubicin, standard drugs used in AML therapy. Isobolograms generated using a range of concentrations of Compound A and cytarabine or daunorubicin in KG-1 cells demonstrated a Combination Index (CI) of less than 1, indicating an overall synergistic interaction (FIG. 7, panels A-B).

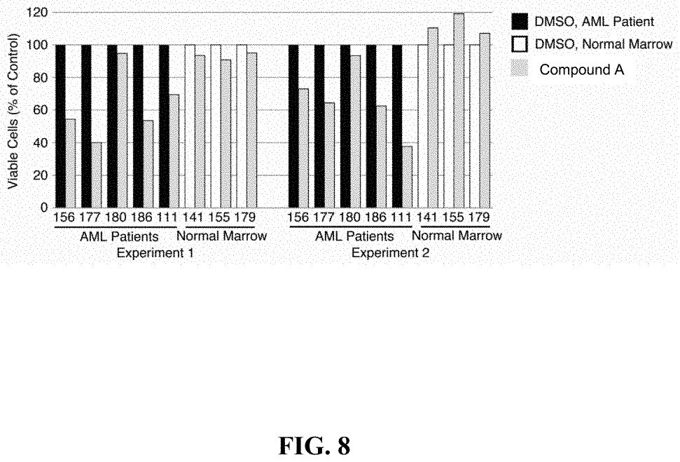

The effects of Compound A were tested on primary AML patient bone marrow samples, obtained at initial diagnosis or at the time of relapse. AML patient samples treated with 2 μM Compound A for 72 hours demonstrated a range of responsiveness, as measured by decreased viable cell count on Trypan Blue exclusion assay (FIG. 2, panel E). AML patient samples with relatively higher CREB expression exhibited a greater loss of viability than those with lower CREB expression (FIG. 2, panel F), indicating that higher CREB expression is associated with greater sensitivity to Compound A, consistent with AML cell line experimental results. In the culture conditions described, AML patient sample cells exhibited no loss in cell viability when treated with 0.1% DMSO during the 72 hour treatment period (FIG. 8). Normal bone marrow samples were treated with Compound A in parallel to assess this agent's potential toxicity to hematopoietic cells. These bone marrow cells expressed nearly undetectable levels of CREB protein, and following 72 hours of treatment with Compound A, the viability of the cells did not significantly change. These studies were extended to methylcellulose colony assays of normal human hematopoietic cells, which also demonstrated no significant decrease in colony formation when treated with up to 10 mM Compound A (FIG. 2, panel G). Together, these data suggest that the potency and efficacy of Compound A in AML cells is dependent on CREB expression, and normal hematopoietic progenitor cells are relatively spared from toxicity.

### Compound A Specifically Disrupts CREB-Driven Transcription in AML Cells

To determine the functional specificity and downstream effects of Compound A in AML, the CREB transcriptome was first defined in KG-1 cells using CREB chromatin immunoprecipitation followed by high-throughput sequencing (CREB ChIP-Se). In parallel, whole transcriptome sequencing (RNA-Seq) of KG-1 cells treated with 5 μM Compound A or 0.1% DMSO for 12 hours was performed to define alterations in the transcription of identified CREB-bound genes. Finally, whole-genomic H3K27 histone acetylation analysis was performed under these same conditions. Acetylation of H3K27 is a function specific to the histone acetyltransferase CBP, and a reduction in acetylation of this lysine residue at CREB-bound loci following Compound A treatment would provide evidence that the CREB-CBP interaction has been successfully disrupted. Together, these three experiments permitted: 1) assembly of a comprehensive catalogue of CREB-bound sites within the AML genome; 2) identification of the set of genes which exhibited altered expression following Compound A treatment, and; 3) measurement of alterations in CBP-mediated histone acetylation at genomic CREB binding sites. In this way, the ‘on-target’ effects of Compound A could be assessed.

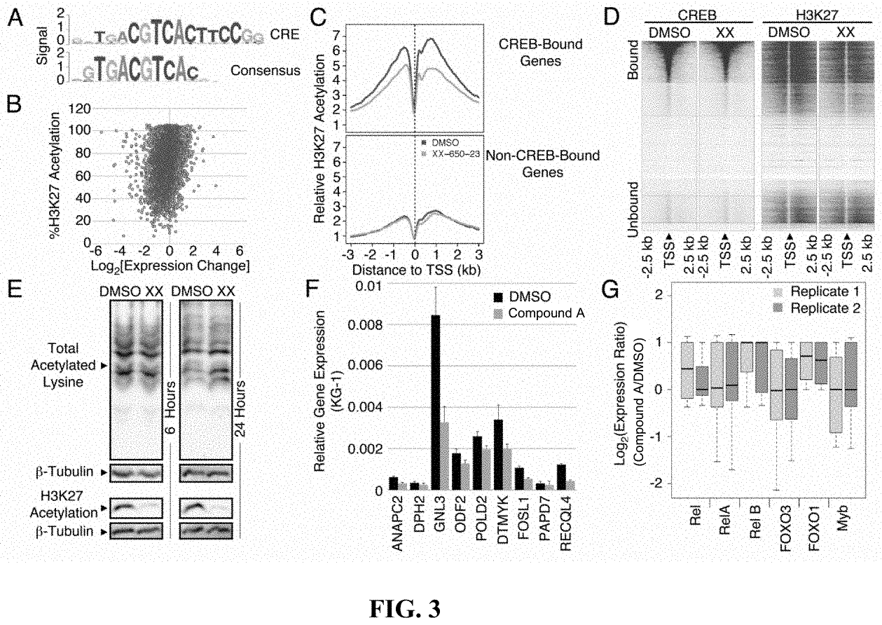

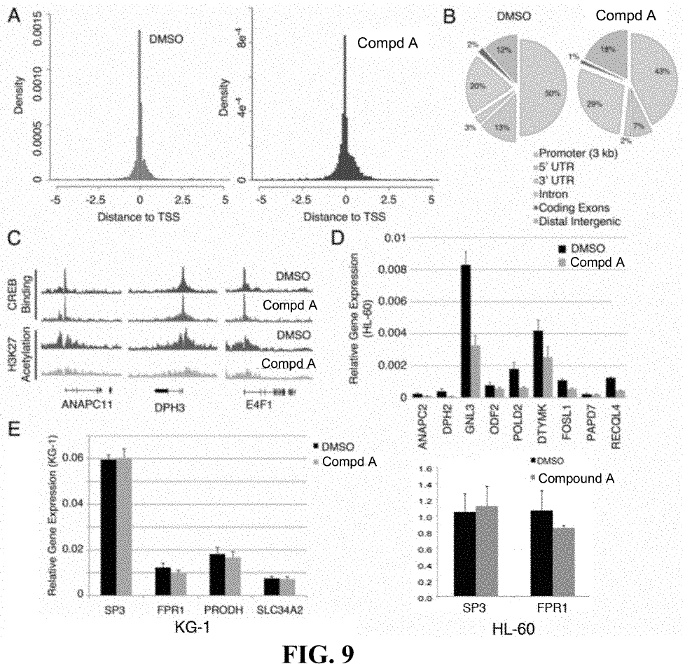

CREB peaks (p-val≤10⁻⁵, fdr≤1%) identified on ChIP-Seq analysis were enriched for the canonical ‘CRE element’ DNA binding sequence (FIG. 3, panel A). CREB binding was detected at 4680 sites in the KG-1 AML cell genome. Of these, 2787 CREB-bound genes exhibited reduced expression following Compound A treatment, with 602 exhibiting greater than 50% reduced expression. Greater than 95% of all CREB-bound genes exhibited decreased H3K27 histone acetylation (FIG. 3, panel B). Within KG-1 cells, >90% of occupied CREB-binding sites (CRE elements) were within 500 bp of a transcription start site (TSS)(FIG. 9, panel A and B). Consistent with this, reductions in CBP-mediated histone acetylation were most pronounced within 1 to 3 kb of these CREB-bound promoter sites (FIG. 3, panel C). Importantly, H3K27 acetylation decreased almost exclusively at CREB-bound loci, indicating specific disruption of the CREB-CBP interaction. CREB genomic binding did not change following Compound A treatment (FIG. 3, panel D and FIG. 9, panel C). Western blot analysis confirmed that Compound A caused a specific decrease in H3K27 acetylation and is not a general inhibitor of acetyltransferase activity (FIG. 3E).

To validate the RNA-Seq data set, qPCR of CREB target genes that exhibited significant (>50%) transcriptional downregulation and reduced H3K27 acetylation was performed in two AML cell lines (KG-1 and HL-60) following treatment with Compound A under conditions identical to those used for RNA-Seq experiments (FIG. 3, panel F and FIG. 9, panel D). Compound A consistently reduced CREB target gene expression in both these cell lines in a statistically significant manner, although we expect that these genes are regulated by other transcription factors. To assess for potential ‘off-target’ effects of Compound A, the RNA-Seq gene expression profiles of other transcription factors that bind CBP, including Rel, RelA, RelB, Foxo3, Foxo1 and Myb, were analyzed. Compound A evoked no significant change in the target gene expression of these other CBP-binding proteins (FIG. 3, panel G). These results were confirmed for a set of Myb-driven genes, SP3, FPR1, PRODH, SLC34A2 in both KG-1 and HL-60 cells (Tapias et al. Transcriptional regulation of the 5-flanking region of the human transcription factor Sp3 gene by NF-1, c-Myb, B-Myb, AP-1 and E2F. Biochim Biophys Acta. 2008; 1779(5):318-29; Xu et al. Transcriptional regulation of the human NaPi-IIb cotransporter by EGF in Caco-2 cells involves c-myb. Am J Physiol Cell Physiol. 2003; 284(5):C1262-71; Pattabiraman et al. Role and potential for therapeutic targeting of MYB in leukemia. Leukemia. 2013; 27(2):269-77; Miettinen H M. Regulation of human formyl peptide receptor 1 synthesis: role of single nucleotide polymorphisms, transcription factors, and inflammatory mediators. PLoS One. 2011; 6(12):e28712) (FIG. 9, panel E). Thus, these results provide evidence that Compound A specifically disrupts the interaction of CBP and CREB, but not that of other CBP-interacting transcription factors in AML cells.

### Compound A Prolongs Survival in Mouse Models of AML Without Toxicity

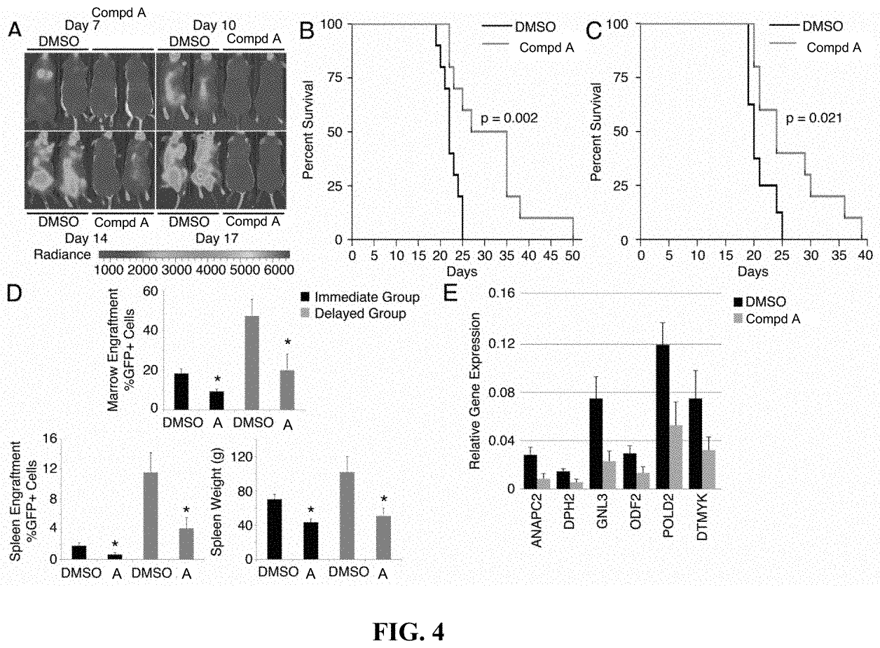

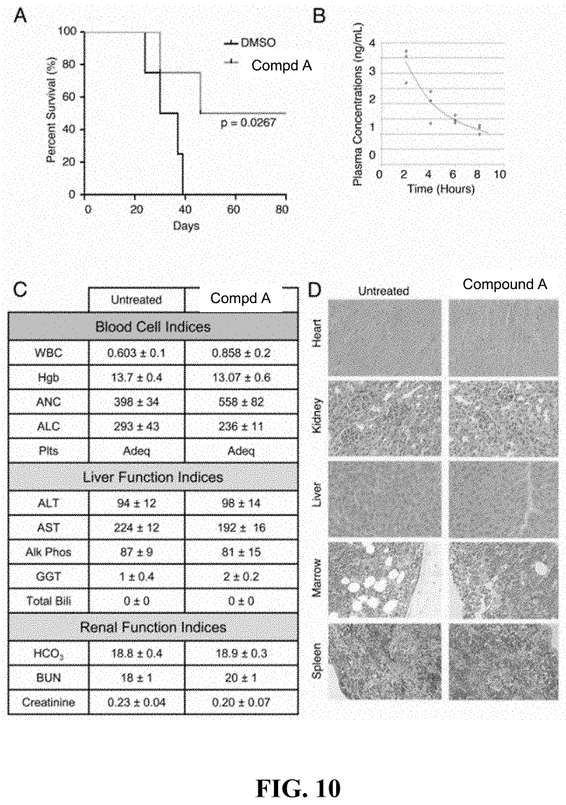

To examine the efficacy of CREB inhibition in an in vivo model of AML, 4-6 week old NOD.Cg-Prkdc^{scid} Il2rg^{tm1Wjl}SzJ (NSG) mice were tail-vein injected with HL-60 AML cells (2×10⁶) expressing Firefly luciferase and GFP. Groups of ten mice received 10% DMSO or 2.3 mg/kg Compound A intravenously (IV) once daily starting the day after cell injection (immediate treatment groups), or one of the same two treatments starting seven days after AML cell injection (delayed treatment groups). This dose of Compound A was selected, as it was the maximum deliverable IV injection concentration and volume in 10% DMSO solution per gram mouse body weight. Bioluminescence imaging performed during treatment revealed less disease burden in delayed-treated mice compared to control (average radiance measurements, given in p/sec/cm²/sr: 5.7×10⁵ for the control group versus 2.8×10⁵ for the Compound A-treated group at day 17) (FIG. 4, panel A). CREB inhibition by Compound A significantly prolonged the median survival in Kaplan-Meier analysis in both the immediate (median survival, 22 days versus 31 days, p=0.002; mean survival 22.3 days vs. 31.2 days) and delayed treatment groups (median survival, 20 days versus 24 days, p=0.021; mean survivals 20.9 vs. 26.4 days) (FIG. 4, panel B and 4, panel C). The efficacy of Compound A was also assessed in a mouse xenograft model using primary AML cell sample (patient sample #186), using similar methods. Mice treated with Compound A demonstrated a significant survival advantage compared to DMSO-treated mice (FIG. 10, panel A).

Endpoint studies were performed to assess AML disease burden at the time of death or sacrifice. Indices of disease burden as measured by GFP expression were significantly lower in each treatment group compared to their respective controls (FIG. 4, panel D). Mice treated with Compound A showed less disease in bone marrow (immediate group, 18.3±2.27% versus 9.3±1.28% GFP+ cells, p=0.011; delayed group 47.2±8.43% versus 19.9±8.22% GFP+ cells, p=0.043) spleen (immediate group, 1.8±0.43% versus 0.64±0.27% GFP+ cells, p=0.019; delayed group, 11.5±2.6% versus 4.1±1.44% GFP+ cells, p=0.042) and in spleen weights (immediate group, 70.1±5.8 mg versus 43.3±3.6 mg, p=0.0025; delayed group, 101.7±18 versus 50.6±9.1 mg, p=0.042). Complete blood counts and blood smear analysis also demonstrated 18-28% circulating myeloblasts in DMSO-treated mice at the time of sacrifice, whereas none of the Compound A-treated mice had detectable circulating leukemia cells (data not shown). In this AML xenograft model, 7 of 10 DMSO-treated mice developed visible visceral tumors (chloromas), which had a mean weight of 0.43±0.15 g, and 4 of 10 mice in the Compound A-treated group developed tumors with a mean weight of 0.027±0.019 g. There were no differences in liver weights across groups (data not shown).

To directly evaluate the effects of Compound A on CREB transcriptional activity in vivo, six NSG mice were injected with 2×10⁶ HL-60 expressing GFP. After a ten-day engraftment period, the mice received three once-daily treatments of either 2.3 mg/kg Compound A or DMSO. The mice were then sacrificed and GFP+ bone marrow cells were sorted and analyzed for transcriptional changes in validated CREB target genes. This experiment recapitulated in vitro findings (FIG. 4, panel E).

Pharmacokinetic studies showed that the half-life of Compound A in plasma is approximately 4.3 hours (FIG. 10, panel B). To assess the potential in vivo toxicities of Compound A, four groups of five mice not injected with AML cells were treated with 2.3 mg/kg Compound A intravenously daily, or 20, 40 or 60 mg/kg intraperitoneally (IP) daily for 28 days. These mice demonstrated normal complete blood counts, liver function tests and kidney function tests, compared to age-matched NSG mice given no treatment (FIG. 10, panel C). Histology demonstrated no microscopic evidence of vital organ damage (FIG. 10, panel D). There was no decrease in animal body weight over 28 days.

### Compound A Induces Apoptosis and Bcl-2 Downregulation

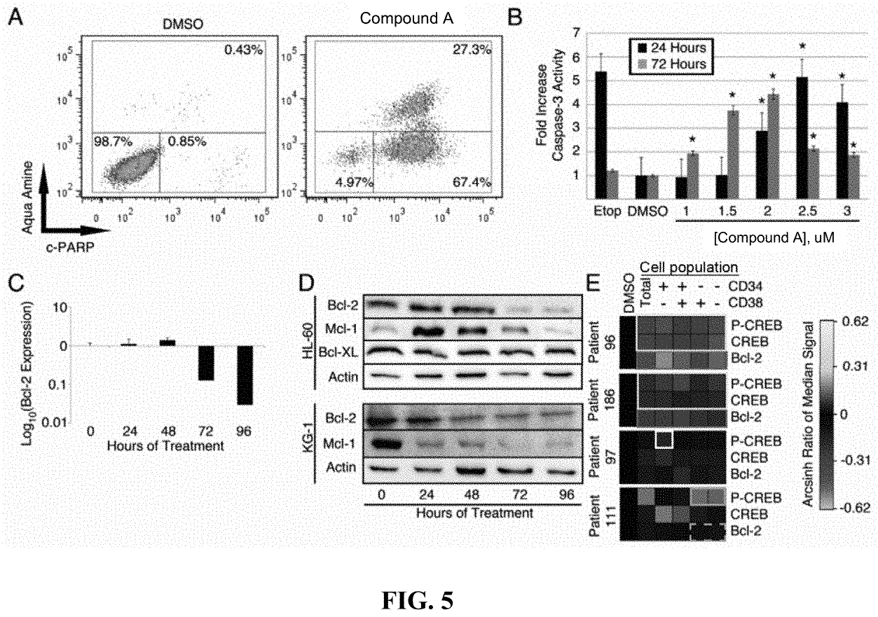

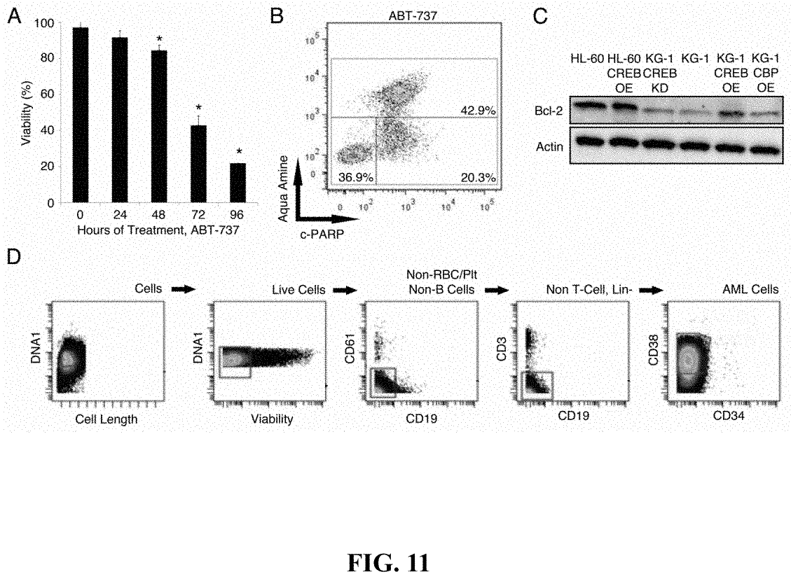

The balance between pro- and anti-apoptotic protein expression contributes to cell fate decisions. CREB regulates the expression of several anti-apoptotic proteins, including Bcl-2, Bcl-XL and Mcl-1. Bcl-2 expression in particular is known to mediate resistance to chemotherapy and influence clinical outcome in a number of cancer types, including AML. The ability of Compound A to cause apoptosis by disrupting the CREB-driven expression of these anti-apoptotic proteins was examined. In HL-60 cells treated with Compound A, apoptosis is induced in a dose- and time-dependent manner. Flow cytometry showed that >95% of cells become apoptotic or are non-viable after 72 hours of treatment with 2 mM Compound A (FIG. 5, panel A). Compound A elicited apoptosis through the intrinsic apoptosis pathway, with activation of Caspase-3 (FIG. 5, panel B) and detectable Caspase-9 cleavage (data not shown). Relative caspase-3 activity at 24 hours: 5.4±0.8 for etoposide, 1±0.78, 0.93±0.71, 1.02±0.67, 2.9±0.58, 5.1±0.63, 4.1±0.71 for 0, 1, 1.5, 2, 2.5 and 3 mM Compound A, respectively. Activity at 72 hours: 1.2±0.05 for etoposide, 1.0±0.02, 1.9±0.03, 3.7±0.04, 4.4±0.05, 2.1±0.03, 1.9±0.02 for 0, 1, 1.5, 2, 2.5 and 3 mM Compound A, respectively. The transcription of Bcl-2 decreased following 72 hours of 2 mM Compound A treatment (FIG. 5, panel C), and this was verified by Western blot analysis (FIG. 5, panel D). In parallel, the expression of Mcl-1 initially increased, then decreased at 72 hours, while Bcl-XL expression remained constant. To confirm whether loss of Bcl-2 function alone is sufficient to induce apoptosis in AML cells, HL-60 cells were treated with the validated Bcl-2 inhibitor ABT-737 (50 to 200 nM). Apoptosis was induced in these cells by ABT-737 after 72 hours of treatment, similar to Compound A (FIG. 11, panels A and B) (Konopleva et al. Mechanisms of apoptosis sensitivity and resistance to the BH3 mimetic ABT-737 in acute myeloid leukemia. Cancer Cell. 2006; 10(5):375-88). KG-1 cells also undergo apoptosis following treatment with Compound A, and similarly exhibited decreased Bcl-2 expression alongside a pronounced decrease in Mcl-1 under these same conditions (FIG. 5, panel D). Bcl-2 levels are influenced by CREB expression in these AML cell lines (FIG. 11, panel C). Together, these data suggest that CREB inhibition causes downregulation of Bcl-2 and Mcl-1 in AML cell lines, and that this represents one mechanism by which Compound A causes apoptosis.

To examine the relationship between Bcl-2, total CREB, and pCREB in primary AML patient samples, we performed single cell multiparameter mass cytometry (CyTOF) analysis on four AML patient samples treated with 2 mM Compound A or 0.1% DMSO for 48 hours. Since disease relapse is thought to occur secondary to survival and adaptation of leukemia stem cells, we wished to specifically examine the effects of CREB inhibition on the CD34+CD38− subpopulations of these AML patient samples. This fraction is reported to contain the putative ‘leukemia initiating’ or ‘stem cell’ population (Bonnet D, and Dick J E. Human acute myeloid leukemia is organized as a hierarchy that originates from a primitive hematopoietic cell. Nat Med. 1997; 3(7):730-7; Majeti R, and Weissman I L. Human acute myelogenous leukemia stem cells revisited: there's more than meets the eye. Cancer Cell. 2011; 19(1):9-10) (see CyTOF gating strategy, FIG. 11, panel D).

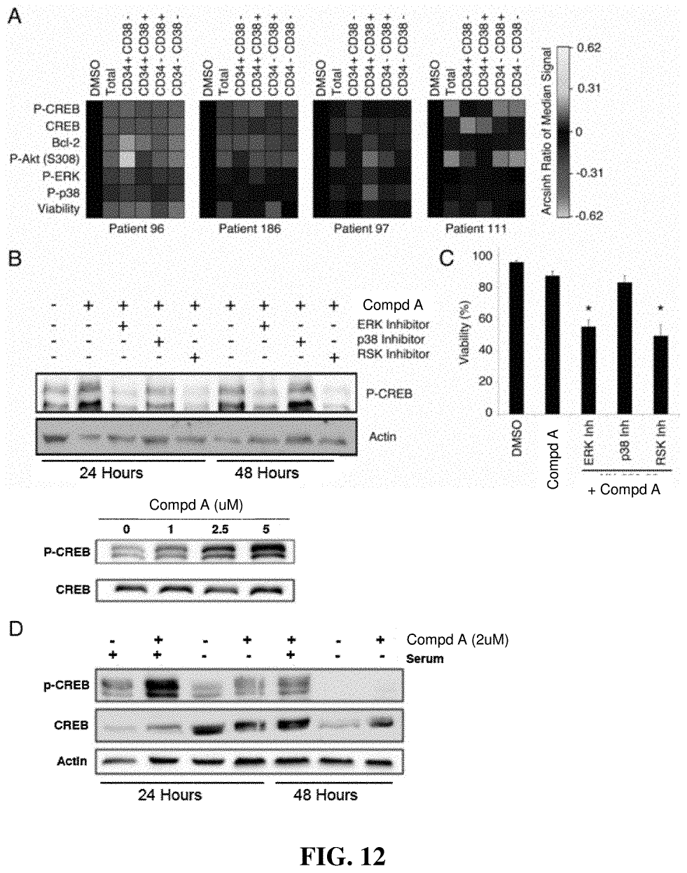

As shown in FIG. 5, panel E, patient samples 96 and 186, which showed relatively higher initial CREB expression and higher responsiveness to Compound A shown in FIG. 2 demonstrated reduced Bcl-2 expression following treatment with Compound A in all cell subpopulations, including the leukemia stem cell-containing population (FIG. 5, panel E, red boxes). This occurred alongside downregulation of total CREB, which regulates its own transcription, as well as reduced phosphorylation at Serine 133, an activating mark and activation of CREB compared to the DMSO treated control (FIG. 5, panel E, yellow boxes). In contrast, patient sample 97, which expressed less CREB at baseline, demonstrated reduced CREB and Bcl-2 expression in all cell subsets except the CD34+CD38− population. In this specific population, CREB phosphorylation was increased (FIG. 5, panel E, white box). These results may indicate that phosphorylation and activation of CREB is sufficient to overcome the competitive blockade imposed by Compound A, and may represent a mechanism of resistance. A similar phenomenon was noted in patient sample 111, obtained from a patient at the time of AML relapse, as increased phosphorylation of CREB was associated with no significant Bcl-2 downregulation in the more mature AML cell populations (FIG. 5, panel E, blue boxes). However, in the leukemia stem cell-containing population (CD34⁺CD38−) of this relapsed AML sample, Bcl-2 downregulation was noted. Extended phenotypic analysis of kinase and signaling pathway activity in these four Compound A-treated samples indicated that the relative activation of AKT and ERK was associated with increases or decreases of phosphorylation of CREB (FIG. 12, panel A). HL-60 cells also show an increase in levels of phosphorylated but not unphosphorylated CREB following 24 hours of Compound A treatment (FIG. 12, panel B), and this effect can be blocked by validated small molecule inhibitors of the ERK and RSK kinases (FIG. 12, panel B), in keeping with previous work describing ERK/RSK-mediated phosphorylation of CREB downstream of the GM-CSF receptor in AML cells (Kwon et al., Granulocyte-macrophage colony-stimulating factor stimulation results in phosphorylation of cAMP response element-binding protein through activation of pp90RSK. Blood. 2000; 95(8):2552-8; Sakamoto et al. Granulocyte-macrophage colony-stimulating factor and interleukin-3 signaling pathways converge on the CREB-binding site in the human egr-1 promoter. Mol Cell Biol. 1994; 14(9):5975-85; Wong A, and Sakamoto K M. Granulocyte-macrophage colony-stimulating factor induces the transcriptional activation of egr-1 through a protein kinase A-independent signaling pathway. J Biol Chem. 1995; 270(51):30271-3). Blockade of these kinases increased the efficacy of Compound A (FIG. 12, panel C). The data with AML cell lines and AML patient samples suggest that CREB activation by ERK and RSK are inhibited by Compound A.

### Compound A Induces AML Cell Cycle Arrest at the G₁/S Transition

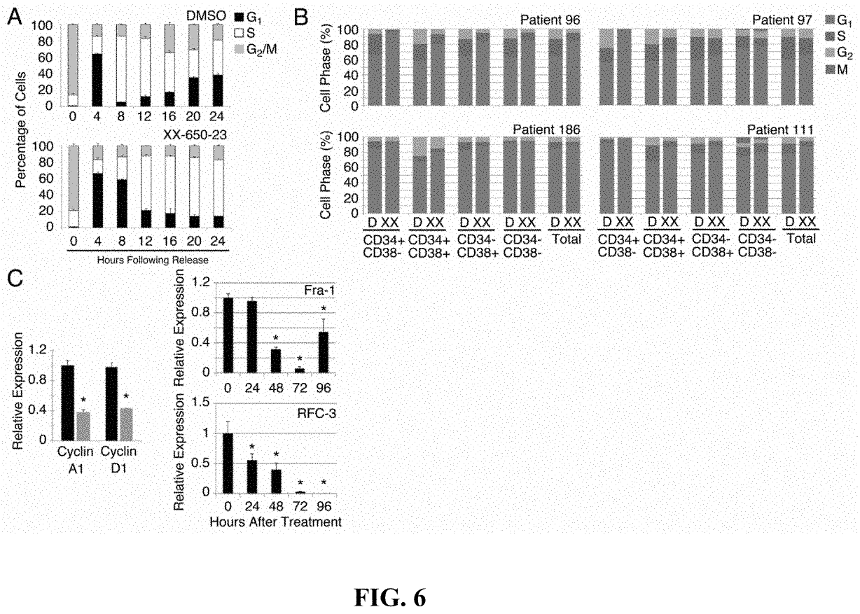

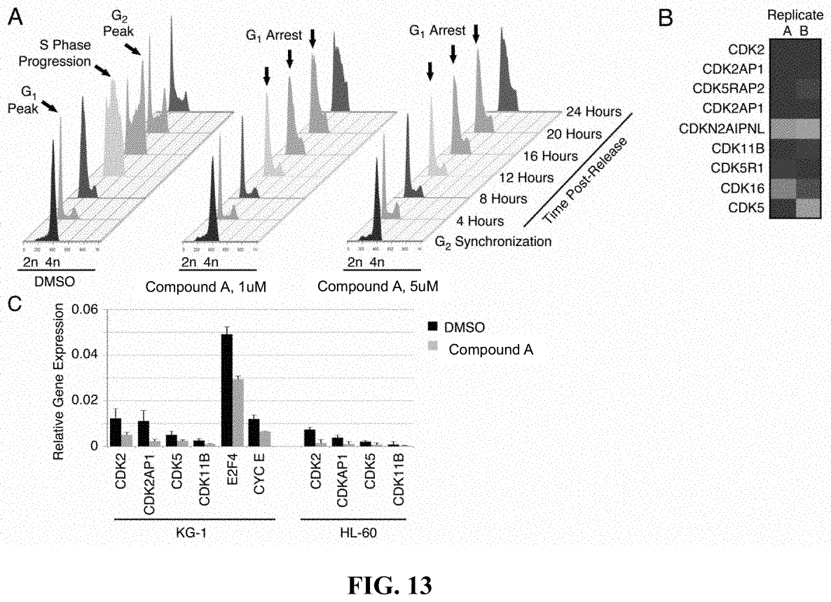

CREB regulates the cell cycle by virtue of transcriptionally regulating the expression of key cell cycle genes. Thus, the effect of Compound A on this aspect of cell function was also examined. Cultured KG-1 cells were synchronized at the G₂ phase using nocodazole (Whitfield et al. Identification of genes periodically expressed in the human cell cycle and their expression in tumors. Mol Biol Cell. 2002; 13(6):1977-2000). Following release, cell cycle progression was followed by DNA content (n) for 24 hours in the presence or absence of Compound A. CREB inhibition caused cell cycle arrest of KG-1 AML cells at the G₁/S transition and also delayed progression through S-phase (% cells in G1-phase for DMSO treated cells: 61.18±0.97% and 4.92±0.33% at 4 and 8 hours, respectively, versus Compound A-treated cells: 66.20±1.83% and 55.41±0.59% at 4 and 8 hours following release, respectively)(FIG. 6, panel A and FIG. 13, panel A). CyTOF cell cycle analysis of AML patient bone marrow samples revealed the same perturbation in the G₁/S transition following Compound A treatment (FIG. 6, panel B). Patient sample 97, which had low CREB expression and only modest loss of viability following Compound A, showed the largest increase in cells arrested in the G₁ phase. This effect was most pronounced in the CD34+CD38− subpopulation, exhibiting virtually no cells in S-phase following treatment (<0.1% of total). In contrast, in patient samples 96 and 186, which displayed higher CREB expression and increased susceptibility to cell death following Compound A treatment, the CD34+CD38+ subpopulation showed the greatest increase in cells in G₁. In all samples and subpopulations, the percent of cells in both the S and G₂/M phases was reduced, and that of G₁ was increased. Thus, AML cells with relatively lower CREB expression may escape cell death caused by Compound A treatment, but cell cycle arrest at the G₁/S transition remains an important effect of CREB inhibition.

To characterize transcriptional changes that could explain AML cell cycle arrest at the G₁/S transition and delayed S-phase progression, alterations in gene programs revealed by RNA-Seq analysis were examined. A number of previously described mediators of the G₁/S transition and S-phase progression were downregulated following Compound A treatment, including a set of CREB-bound cyclin-dependent kinases, Cyclin A, and the coordinately functioning pair Cyclin D1 and Fra-1(Boulon et al. Oct-1 potentiates CREB-driven cyclin D1 promoter activation via a phospho-CREB- and CREB binding protein-independent mechanism. Mol Cell Biol. 2002; 22(22):7769-79; Desdouets et al. Cell cycle regulation of cyclin A gene expression by the cyclic AMP-responsive transcription factors CREB and CREM. Mol Cell Biol. 1995; 15(6):3301-9; Burch et al. An extracellular signal-regulated kinase 1- and 2-dependent program of chromatin trafficking of c-Fos and Fra-1 is required for cyclin D1 expression during cell cycle reentry. Mol Cell Biol. 2004; 24(11):4696-709) (FIG. 6, panel C). RNA-Seq and CREB ChIP-Seq also revealed downregulation of an additional set of cyclins, and a subset of these transcriptional alterations were confirmed by qPCR in two AML cell lines (FIG. 13, panels B and C). In addition, Replication Factor C3 (RFC-3), a member of the PCNA DNA replication complex, is downregulated following CREB knockdown in AML cells, which is associated with G₁/S transition arrest (Chae et al. Replication factor C3 is a CREB target gene that regulates cell cycle progression through the modulation of chromatin loading of PCNA. Leukemia. 2015; 29(6):1379-89). Levels of RFC-3 also decrease following Compound A treatment (FIG. 6, panel C).

Targeting the activity of specific transcription factors for the treatment of leukemia has begun to show promise in a number of pre-clinical studies. The interaction of CBP with b- and g-catenin has recently been targeted using a small molecule, and this strategy was effective against both primary and relapsed ALL (Gang et al. Small-molecule inhibition of CBP/catenin interactions eliminates drug-resistant clones in acute lymphoblastic leukemia. Oncogene. 2013). Another group recently demonstrated the efficacy of targeting the mutant fusion transcription factor CBPb-SMMHC, which drives inv(16)+ AML (Illendula et al. Chemical biology. A small-molecule inhibitor of the aberrant transcription factor CBFbeta-SMMHC delays leukemia in mice. Science. 2015; 347(6223):779-84), and the critical interaction between menin and MLL fusion proteins, which drives subtypes of both AML and ALL, has also been successfully targeted (Grembecka et al. Menin-MLL inhibitors reverse oncogenic activity of MLL fusion proteins in leukemia. Nat Chem Biol. 2012; 8(3):277-84). These pre-clinical studies demonstrate the potential of transcription factor-directed therapy, and encourage further development of novel candidate compounds for eventual clinical use. The data presented here similarly provide “proof of principle” that CREB can be targeted for the treatment of AML and a variety of other cancers, and lay the groundwork for advancing this strategy to the clinical arena.

### Example 3

The compounds prepared according to the methods described herein and tested for activity in the CellTiter Glo assay in HL60 (Human promyelocytic leukemia cell line), Jukat (human T lymphocyte cell line for acute T cell leukemia) and/or Nalm6 (human ALL cell line) cells. Selected results are summarized in Table 4.

TABLE 4
Comparison of selected compounds
in the CellTiter Glo assay in
HL60 cells. IC₅₀ measured as A = < 1 uM;
B = 1-10 uM; C = > 10 uM

| Compound | HL60 | Jukat | Nalm6 |
|---|---|---|---|
| # | IC₅₀ (uM) | IC₅₀ (uM) | IC₅₀ (uM) |
| 1 | B | | |
| 2 | A | | |
| 3 | A | | |
| 5 | A | | |
| 6 | A | | |
| 7 | A | | |
| 8 | A | | |
| 9 | A | | |
| 10 | A | | |
| 11 | A | | |
| 12 | A | | |
| 13 | A | | |
| 15 | B | A | B |
| 16 | A | | |
| 17 | A | A | A |
| 19 | B | | |
| 20 | B | B | B |
| 21 | A | | |
| 22 | A | | |
| 23 | A | A | A |
| 24 | A | A | A |
| 25 | A | | |
| 26 | A | | |
| 28 | A | A | |
| 27 | A | A | |
| 29 | A | A | A |
| 30 | A | | |
| 31 | A | A | B |
| 32 | A | A | B |
| 33 | B | B | B |
| 34 | A | | |
| 35 | A | | |
| 36 | B | | |
| 37 | B | A | B |
| 38 | A | C | B |
| 39 | B | B | B |
| 40 | B | B | B |
| 41 | A | | |
| 42 | A | A | B |
| 43 | B | B | B |
| 44 | A | A | A |
| 45 | A | A | B |
| 46 | A | A | A |
| 47 | A | C | A |
| 48 | A | A | B |
| 49 | A | B | B |
| 50 | A | A | B |
| 51 | A | A | A |
| 52 | A | A | B |
| 53 | B | | |
| 54 | B | C | C |
| 55 | B | | |
| 56 | B | A | B |
| 57 | B | B | B |
| 58 | A | B | B |
| 61 | B | | |
| 62 | B | | |
| 63 | A | A | B |
| 101 | A | A | A |
| 159 | A | A | A |
| 160 | A | | |
| 161 | A | A | C |
| 233 | B | B | B |

### Table 5

Compounds were prepared according to the methods described herein and tested for activity in the CellTiter Glo assay in HL60 (Human promyelocytic leukemia cell line), Jukat (human T lymphocyte cell line for acute Tcell leukemia) and/or Nalm6 (human ALL cell line) cells. Selected results are summarized in Table 5.

TABLE 5
Comparison of selected compounds
in the CellTiter Glo assay
in various cells. IC₅₀ measured
as A = < 100 nM; B = 100 nM-1 uM,
C = 1-10 uM; D => 10 uM

| Compound | HL60 | Jukat | Nalm6 |
|---|---|---|---|
| # | IC₅₀ (uM) | IC₅₀ (uM) | IC₅₀ (uM) |
| 64 | B | B | A |
| 65 | B | B | B |
| 66 | B | B | B |
| 67 | B | B | B |
| 68 | B | B | B |
| 69 | B | B | B |
| 70 | C | B | C |
| 71 | B | A | A |
| 72 | B | B | B |
| 73 | C | B | B |
| 74 | B | | |
| 75 | B | | |
| 76 | B | B | A |
| 77 | B | B | B |
| 78 | B | B | A |
| 79 | C | C | C |
| 80 | B | B | B |
| 81 | B | B | B |
| 82 | B | B | A |
| 83 | C | B | B |
| 84 | B | A | B |
| 85 | B | B | B |
| 86 | C | B | B |
| 87 | B | | |
| 88 | B | | |
| 89 | C | | |

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the embodiments shown and described herein. Rather, the scope and spirit of present invention is embodied by the appended embodiments.

Notwithstanding the appended claims, the disclosure set forth herein is also described by the following clauses:

Clause 1. A method for modulating transcription of CREB in a cell that overexpresses CREB, the method comprising:

contacting the cell with an effective amount of a CREB transcription inhibitor to modulate transcription of CREB in the cell, wherein the inhibitor is described by formula (I):

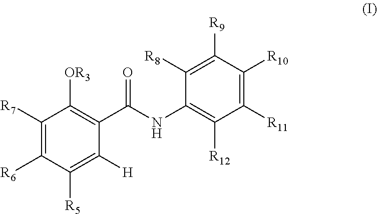

wherein:

R₃ is H;

R₈, R₉, R₁₀, R₁₁ and R₁₂ are independently selected from H, halogen, alkyl, substituted alkyl, alkoxy, substituted alkoxy, an electron withdrawing group (e.g., cyano, nitro, trifluoromethyl, etc), phenyl, substituted phenyl, substituted amino, carboxy ester (e.g., CO₂R where R is alkyl or substituted alkyl); and

R₅, R₆ and R₇ are independently selected from H, F, Cl, Br, alkyl, substituted alkyl, aryl, substituted aryl, heteroaryl, substituted heteroaryl, an electron withdrawing group (e.g., cyano, nitro, trifluoromethyl, etc), alkoxy and substituted alkoxy, wherein optionally R₆ and R₇ or R₅ and R₆ are cyclically linked to form a fused aryl or heteroaryl ring which is optionally further substituted; or a salt thereof, or a prodrug form thereof.

Clause 2. The method of clause 1, wherein the inhibitor specifically binds the KIX domain of CREB Binding Protein (CBP).
Clause 3. The method of any one of clauses 1-2, wherein the inhibitor has one of formulae (II)-(IV):

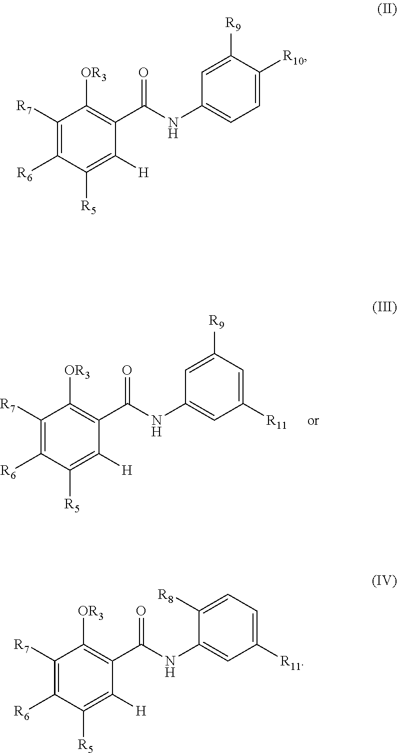

Clause 4. The method of clause 3, wherein the inhibitor is of formula (II) and has one of formulae (V)-(VIII):

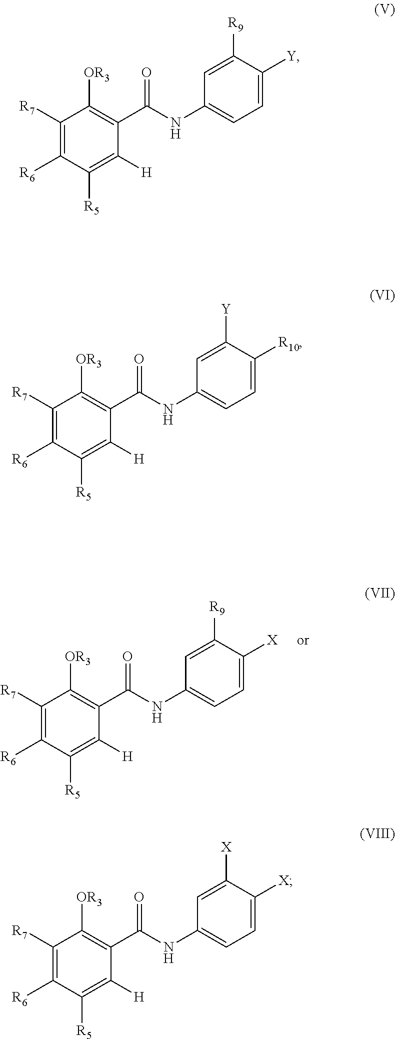

wherein: Y is an electron withdrawing group (e.g., cyano, nitro or trifluoromethyl); and X is a halogen (e.g., Cl, Br or F).
Clause 5. The method of clause 3, wherein the inhibitor is of formula (III) and has one of formulae (IX)-(XII):

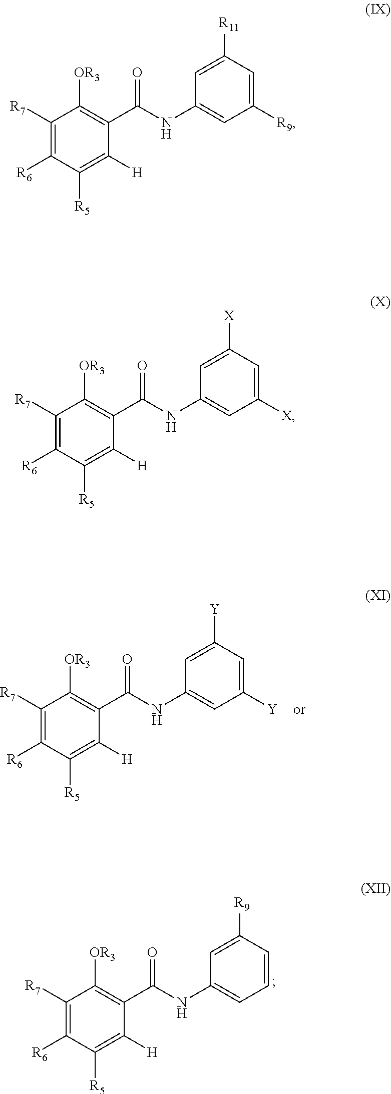

wherein: Y is an electron withdrawing group (e.g., cyano, nitro or trifluoromethyl); and X is a halogen (e.g., Cl, Br or F).
Clause 6. The method of clause 3, wherein the inhibitor is of formula (IV) and has one of formulae(XIII)-(XV):

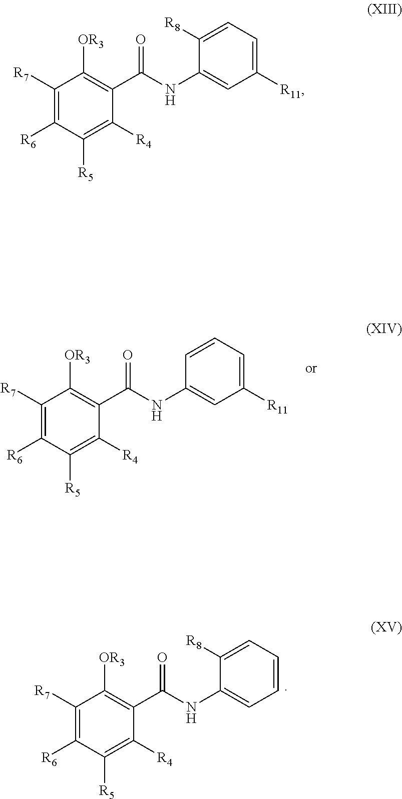

Clause 7. The method of any one of clauses 1-2, wherein the inhibitor is of formula (XVI):

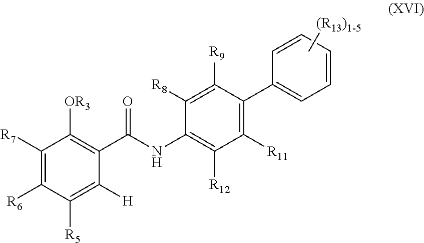

wherein each R₁₃ is independently selected from H, halogen, alkyl, substituted alkyl, alkoxy, substituted alkoxy, hydroxy, cyano, nitro, aryl, substituted aryl, heterocycle, substituted heterocycle, heteroaryl, substituted heteroaryl, amino, substituted amino, carboxy, carboxy ester (e.g., CO₂R where R is alkyl or substituted alkyl), sulfonyl, sulfonate, sulfonamide and substituted sulfonamide.
Clause 8. The method of any one of clauses 1-7, wherein R₅ is halogen (e.g., Cl, Br or F).
Clause 9. The method of any one of clauses 1-8, wherein R₆ and R₇ are each hydrogen.
Clause 10. The method of any one of clauses 1-8, wherein R₆ is halogen (e.g., Cl, Br or F).
Clause 11. The method of any one of clauses 1-8, wherein R₆ is an electron withdrawing group (e.g., CN, NO₂ or CF₃).
Clause 12. The method of clause 10 or 11, wherein R₅ and R₇ are each hydrogen.
Clause 13. The method of any one of clauses 1-8, wherein R₇ is halogen (e.g., Cl, Br or F).
Clause 14. The method of clause 13, wherein R₅ and R₆ are each hydrogen.
Clause 15. The method of any one of clauses 1-7, wherein R₅ or R₆ is an aryl, a substituted aryl, a heteroaryl or a substituted heteroaryl.
Clause 16. The method of clause 15, wherein the inhibitor has one of formulae (XVIIa)-(XVIIIa):

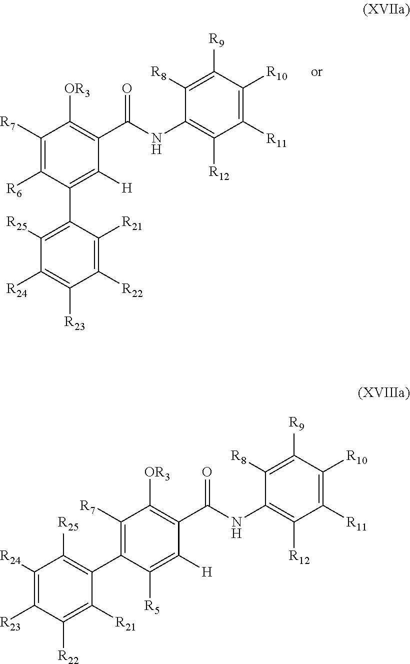

wherein R₂₁-R₂₅ are independently selected from H, halogen, alkyl, substituted alkyl, alkoxy, substituted alkoxy, hydroxy, cyano, nitro, aryl, substituted aryl, heterocycle, substituted heterocycle, heteroaryl, substituted heteroaryl, amino, substituted amino, carboxy, carboxy ester (e.g., CO₂R where R is alkyl or substituted alkyl), sulfonyl, sulfonate, sulfonamide and substituted sulfonamide.
Clause 17. The method of clause 16, wherein the inhibitor is a compound of Table 1 or 2.
Clause 18. The method of any one of clauses 1-7, wherein the inhibitor has one of formulae (XIX)-(XXI):

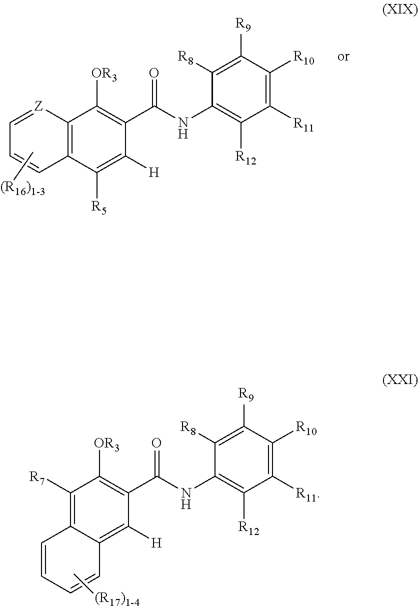

wherein: Z is CR₁₆ or N; and each R₁₆ and each R₁₇ is independently selected from H, halogen, alkyl, substituted alkyl, alkoxy, substituted alkoxy, hydroxy, cyano, nitro, aryl, substituted aryl, heterocycle, substituted heterocycle, heteroaryl, substituted heteroaryl, amino, substituted amino, carboxy, carboxy ester (e.g., CO₂R where R is alkyl or substituted alkyl), sulfonyl, sulfonate, sulfonamide and substituted sulfonamide.
Clause 19. The method of clause 18, wherein the inhibitor is of formula (XIX) and wherein: Z is N; and R₁₀ is cyano, trifluoromethyl or halogen.
Clause 20. The method of clause 18, wherein the inhibitor is of formula (XIX) and wherein: Z is N; and R₉ and R₁₀ are independently halogen.
Clause 21. The method of clause 18, wherein the inhibitor is of formula (XIX) and wherein: Z is N; and R₉ and R₁ are independently halogen or trifluoromethyl.
Clause 22. The method of any one of clauses 1-21, wherein R₅ is H, halogen, aryl (e.g., phenyl) or heterocycle (e.g., 2-furanyl).
Clause 23. The method of clause 18, wherein the inhibitor is a compound of Table 4.
Clause 24. The method of any one of clauses 1-14, wherein R₃ and R5-R₁₂ are selected from one of the following combinations:

| Combination | R₃ | R₅ | R₆ | R₇ | R₈ | R₉ | R₁₀ | R₁₁ | R₁₂ |
|---|---|---|---|---|---|---|---|---|---|
| 1 | H | H | H | H | H | H | CN | H | H |
| 2 | H | Br | H | H | H | H | CN | H | H |
| 3 | H | Cl | H | H | H | H | CN | H | H |
| 4 | H | Cl | H | H | H | Cl | CN | H | H |
| 5 | H | F | H | H | H | Cl | CN | H | H |
| 6 | H | F | H | H | H | Me | CN | H | H |
| 7 | H | Br | H | H | H | Cl | CN | H | H |
| 8 | H | F | H | H | H | H | CN | H | H |
| 9 | H | H | Br | H | H | H | CN | H | H |
| 10 | H | H | F | H | H | H | CN | H | H |
| 11 | H | H | Br | H | Cl | H | CN | H | H |
| 12 | H | H | Br | H | F | H | CN | H | H |
| 13 | H | H | Br | H | H | Cl | CN | H | H |
| 14 | H | H | H | Br | H | H | CN | H | H |
| 15 | H | Ph | H | H | H | H | CN | H | H |
| 16 | H | H | Cl | H | H | H | CN | H | H |
| 17 | H | H | CF₃ | H | H | H | CN | H | H |
| 18 | H | H | CN | H | H | H | CN | H | H |
| 19 | H | F | H | H | H | CN | Me | H | H |
| 20 | H | F | H | H | H | CN | F | H | H |
| 21 | H | F | H | H | H | Me | NO₂ | H | H |
| 22 | H | F | H | H | Cl | H | NO₂ | H | H |
| 23 | H | Cl | H | H | Cl | H | NO₂ | H | H |
| 24 | Ac | Cl | H | H | Cl | H | NO₂ | H | H |
| 25 | H | Cl | H | H | H | Me | NO₂ | H | H |
| 26 | H | F | H | H | H | Cl | Br | H | H |
| 27 | Ac | F | H | H | H | Cl | Br | H | H |
| 28 | C₇H₁₅CO | F | H | H | H | Cl | Br | H | H |
| 29 | 3-methyl-butanoyl | F | H | H | H | Cl | Br | H | H |
| 30 | H | Cl | H | H | H | Cl | Br | H | H |
| 31 | H | F | H | H | H | Cl | Cl | H | H |
| 32 | H | F | H | H | H | F | F | H | H |
| 33 | H | F | H | H | H | F | H | H | H |
| 34 | H | F | H | H | H | Me | Cl | H | H |
| 35 | H | Cl | H | H | H | Me | Cl | H | H |
| 36 | H | F | H | H | Me | H | Cl | H | H |
| 37 | H | F | H | H | F | H | Cl | H | H |
| 38 | H | F | H | H | Cl | H | Cl | H | H |
| 39 | H | F | H | H | F | H | F | H | H |
| 40 | H | F | H | H | F | F | H | H | H |
| 41 | H | F | H | H | H | F | H | F | H |
| 42 | H | F | H | H | H | Cl | H | Cl | H |
| 43 | H | F | H | H | H | Cl | H | H | H |
| 44 | H | F | H | H | H | H | CF₃ | H | H |
| 45 | H | F | H | H | H | H | OCF₃ | H | H |
| 46 | H | F | H | H | H | Cl | OCF₃ | H | H |
| 47 | H | F | H | H | H | CF₃ | Cl | H | H |
| 48 | H | F | H | H | H | CF₃ | F | H | H |
| 49 | H | F | H | H | H | CF₃ | Me | H | H |
| 50 | H | F | H | H | H | CF₃ | H | H | H |
| 51 | H | F | H | H | H | CF₃ | H | CF₃ | H |
| 52 | H | F | H | H | H | OCF₃ | H | H | H |
| 53 | H | F | H | H | H | H | Ph | H | H |
| 54 | H | F | H | H | H | H | OMe | H | H |
| 55 | H | F | H | H | H | OMe | F | H | H |
| 56 | H | F | H | H | H | Me | F | H | H |
| 57 | H | F | H | H | H | H | CO₂Et | H | H |
| 58 | H | F | H | H | H | H | H | CO₂Et | H |
| 59 | H | F | H | H | H | H | H | NMe₂ | H |
| 60 | H | F | H | H | Cl | H | H | H | H |
| 61 | H | F | H | H | F | H | H | H | H |
| 62 | H | F | H | H | CF₃ | H | H | H | H |
| 63 | H | F | H | H | Me | H | H | CF₃ | H |
| 151 | H | Cl | H | H | H | Br | Cl | H | H |
| 152 | H | Cl | H | H | H | H | CF₃ | H | H |
| 153 | H | Cl | H | H | H | Cl | Br | H | CH₃ |
| 154 | H | Cl | H | H | H | H | CF₃ | H | CH₃ |
| 155 | H | Br | H | H | H | Br | Cl | H | H |
| 156 | H | Br | H | H | H | H | CF₃ | H | H |
| 157 | H | Br | H | H | H | Cl | Br | H | CH₃ |
| 158 | H | Br | H | H | H | H | CF₃ | H | CH₃ |
| 159 | H | Br | H | H | H | CF₃ | H | CF₃ | H |
| 160 | H | F | H | H | H | H | NO₂ | H | H |
| 161 | H | Cl | F | H | H | H | CN | H | H |

Clause 25. The method of any one of clauses 1-24, wherein the inhibitor is a prodrug form of the compound, wherein the prodrug form is selected from an acyl, substituted acyl, phosphate ester, or a [1,3]oxazine-2,4(3)-dione derivative of the compound.
Clause 26. A method for inhibiting the proliferation of a cancer cell, in an individual in need thereof the method comprising: contacting a cancer cell with an effective amount of a CREB transcription inhibitor compound (eg. as described herein such as a compound recited in one of clauses 1-25) to inhibit proliferation of the cell.
Clause 27. The method of clause 26, wherein the cell is an Acute Myeloid Leukemia (AML) cell.
Clause 28. The method of clause 26, wherein the cell is an Acute Lymphomblastic Leukemia (ALL) cell.
Clause 29. A method for alleviating symptoms associated with cancer (e.g., a hematologic cancer, such as a leukemia) in a subject in need thereof, the method comprising: administering to the subject an effective amount of a CREB transcription inhibitor compound (e.g., as described herein, such as a compound recited in one of clauses 1-25 and 33-46) to alleviate at least one symptom associated with cancer in the individual.
Clause 30. The method of clause 29, wherein administration of the inhibitor compound alleviates at least one symptom associated with Acute Myeloid Leukemia (AML).
Clause 31. The method of clause 29, wherein administration of the inhibitor compound alleviates at least one symptom associated with Acute Lymphomblastic Leukemia (ALL).
Clause 32. The method of any one of clauses 29-31, wherein the at least one symptom is selected from headache, dizziness or lightheadedness, chest pain, weakness, fainting, vision changes, numbness or tingling of extremities, redness, throbbing or burning pain in extremities (erythromelalgia), enlarged spleen, nosebleeds, bruising, bleeding from mouth or gums, bloody stool and stroke.
Clause 33. A CREB transcription inhibitor compound having one of formulae (II)-(IV):

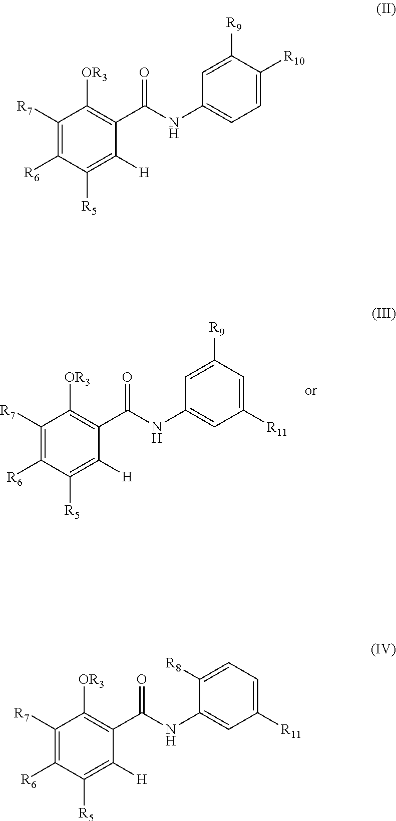

wherein:

R₃ is selected from H an acyl;

R₈, R₉, R₁₀ and R are independently selected from H, halogen, alkyl, substituted alkyl, alkoxy, substituted alkoxy, an electron withdrawing group (e.g., cyano, nitro, trifluoromethyl, etc), phenyl, substituted phenyl, substituted amino, carboxy ester (e.g., CO₂R where R is alkyl or substituted alkyl); and

R₅, R₆ and R₇ are independently selected from H, F, Cl, Br, alkyl, substituted alkyl, an electron withdrawing group (e.g., cyano, nitro, trifluoromethyl, etc), alkoxy and substituted alkoxy, wherein optionally R₆ and R₇ or R₅ and R₆ are cyclically linked to form a fused aryl or heteroaryl ring which is optionally further substituted.

Clause 34. The inhibitor of clause 33, wherein R₅ is halogen (e.g., Cl, Br or F).
Clause 35. The inhibitor of clause 33 or 34, wherein R₆ and R₇ are each hydrogen.
Clause 36. The inhibitor of clause 33, wherein R₆ is halogen (e.g., Cl, Br or F).
Clause 37. The inhibitor of clause 33, wherein R₆ is an electron withdrawing group (e.g., CN, NO₂ or CF₃).
Clause 38. The inhibitor of clause 33, 36 or 37, wherein R₅ and R₇ are each hydrogen.
Clause 39. The inhibitor of clause 33, wherein R₇ is halogen (e.g., Cl, Br or F).
Clause 40. The inhibitor of clause 39, wherein R₅ and R₆ are each hydrogen.
Clause 41. The inhibitor of clause 33, wherein R₅ or R₆ is an aryl, a substituted aryl, a heteroaryl or a substituted heteroaryl.
Clause 42. The inhibitor of clause 41, wherein the inhibitor has one of formulae (XVIIa)-(XVIIIb):

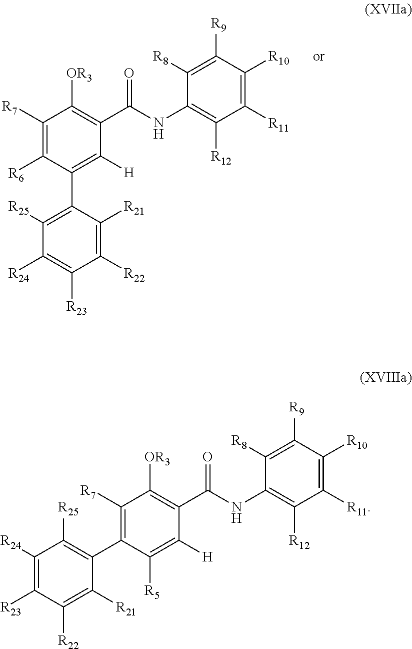

Clause 43. The inhibitor of clause 42, wherein the inhibitor is a compound of Table 1 or 2.
Clause 44. The inhibitor of clause 33, wherein the inhibitor has one of formulae (XIX)-(XXI):

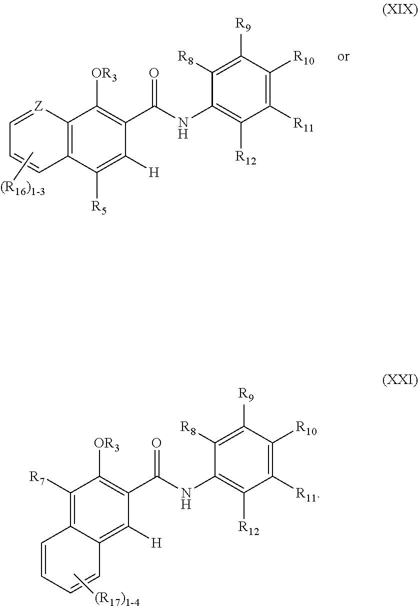

wherein:

Z is CR₁₆ or N; and

each R₁₆ and each R₁₇ is independently H, halogen, alkyl, substituted alkyl, nitro, hydroxy, alkoxy, substituted alkoxy, carboxy, carbonyloxyalkyl, carbonyloxy(substituted alkyl), aryl, substituted aryl, heteroaryl, substituted heteroaryl, amino and substituted amino.

Clause 45. The inhibitor of clause 44, wherein the inhibitor is of formula (XIX) and wherein: Z is N; and R₁₀ is cyano, trifluoromethyl or halogen.
Clause 46. The inhibitor of clause 44, wherein the inhibitor is of formula (XIX) and wherein: Z is N; and R₉ and R₁₀ are independently halogen.
Clause 47. The inhibitor of clause 44, wherein the inhibitor is of formula (XIX) and wherein: Z is N; and R₉ and R₁₁ are independently halogen or trifluoromethyl.
Clause 48. The inhibitor of any one of clauses 44-47, wherein R₅ is H, halogen, aryl (e.g., phenyl) or heterocycle (e.g., 2-furanyl).
Clause 49. The inhibitor of clause 33, wherein the inhibitor is a compound of Table 4.
Clause 50. The inhibitor of clause 33, wherein the inhibitor compound is of Table 3.
Clause 51. The inhibitor of any one of clauses 33-50, wherein the inhibitor is a prodrug form of the compound, wherein the prodrug form is selected from an acyl, substituted acyl, phosphate ester, or a [1,3]oxazine-2,4(3H)-dione derivative of the compound.
Clause 52. A pharmaceutical composition, comprising: a CREB transcription inhibitor compound according to any one of clauses 33-51; and a pharmaceutically acceptable excient.
Clause 53. Use of an inhibitor compound according to any one of clauses 33-51 in medicine.
Clause 54. Use of an inhibitor compound according to any one of clauses 33-51 for treating cancer.
Clause 55. Use of an inhibitor compound according to any one of clauses 33-51 for treating a hematologic cancer.
Clause 56. Use of an inhibitor compound according to any one of clauses 33-51 for treating Acute Myeloid Leukemia (AML) or Acute Lymphomblastic Leukemia (ALL).

1. A method for modulating transcription of CREB in a cell that overexpresses CREB, the method comprising:
contacting the cell with an effective amount of a CREB transcription inhibitor to modulate transcription of CREB in the cell, wherein the inhibitor is described by formula (I): 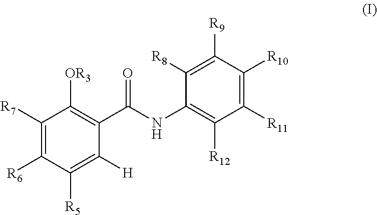
wherein:
R₃ is H;
R₈, R₉, R₁₀, R₁₁ and R₁₂ are independently selected from H, halogen, alkyl, substituted alkyl, alkoxy, substituted alkoxy, an electron withdrawing group, phenyl, substituted phenyl, substituted amino, carboxy ester; and
R₅, R₆ and R₇ are independently selected from H, F, Cl, Br, alkyl, substituted alkyl, aryl, substituted aryl, heteroaryl, substituted heteroaryl, an electron withdrawing group, alkoxy and substituted alkoxy, wherein optionally R₆ and R₇ or R₅ and R₆ are cyclically linked to form a fused aryl or heteroaryl ring which is optionally further substituted;
or a salt thereof, or a prodrug form thereof.

2. The method of claim 1, wherein the inhibitor has one of formulae (II)-(IV):

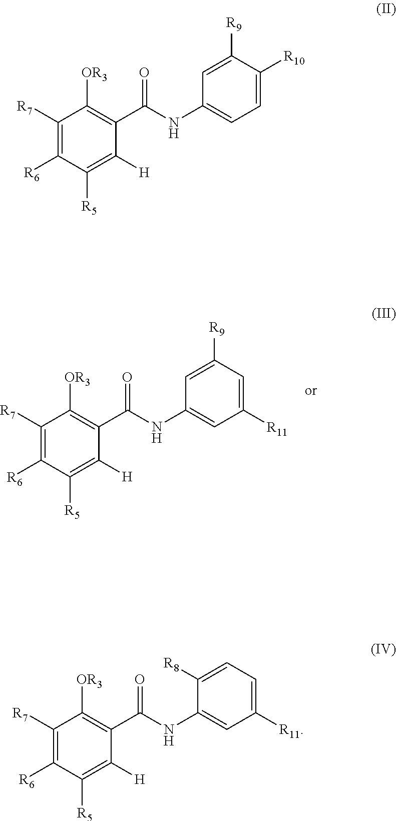

3. The method of claim 2, wherein the inhibitor is of formula (II) and has one of formulae (V)-(VII): 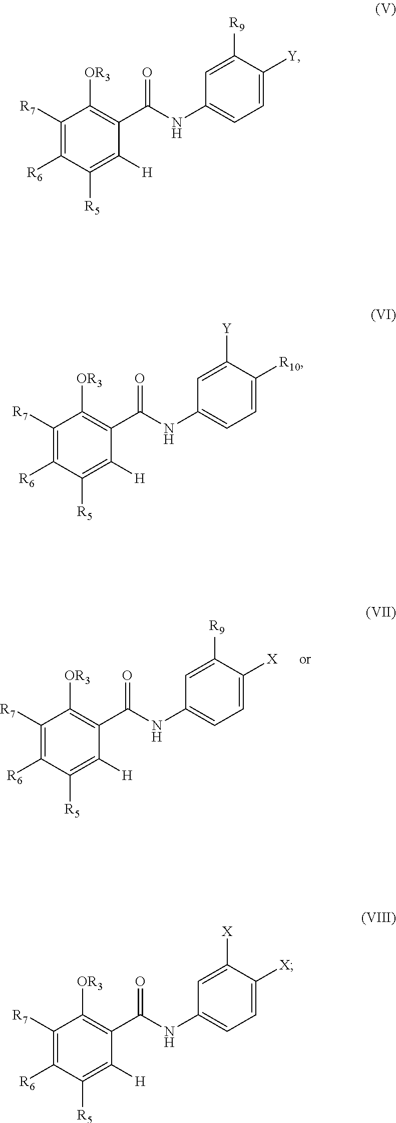
wherein:
Y is an electron withdrawing group; and
X is a halogen.

4. The method of claim 2, wherein the inhibitor is of formula (III) and has one of formulae (IX)-(XII): 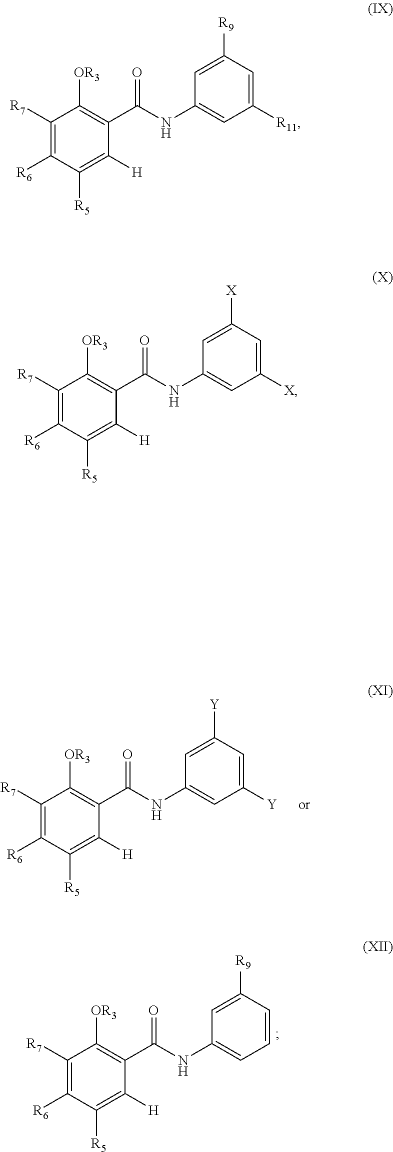
wherein:
Y is an electron withdrawing group; and
X is a halogen.

5. (canceled)

6. (canceled)

7. The method of claim 1, wherein the inhibitor has one of formulae (XVIIa)-(XVIIIa): 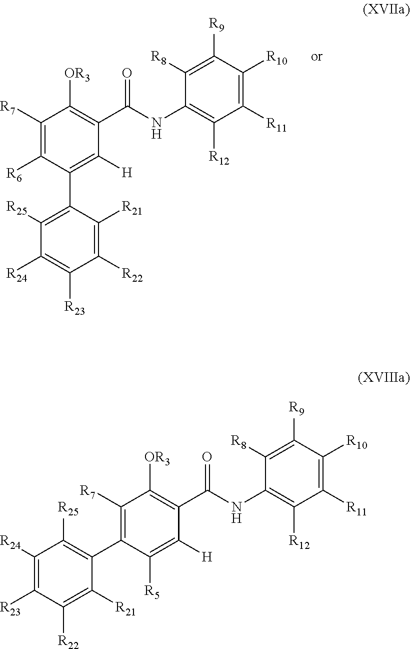
wherein R₂₁-R₂₅ are independently selected from H, halogen, alkyl, substituted alkyl, alkoxy, substituted alkoxy, hydroxy, cyano, nitro, aryl, substituted aryl, heterocycle, substituted heterocycle, heteroaryl, substituted heteroaryl, amino, substituted amino, carboxy, carboxy ester, sulfonyl, sulfonate, sulfonamide and substituted sulfonamide.

8. The method of claim 1, wherein the inhibitor is a compound of Table 1 or 2.

9. The method of claim 1, wherein the inhibitor has one of formulae (XIX)-(XXI): 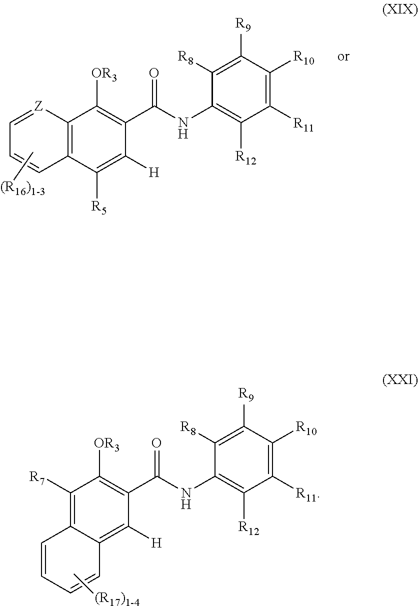
wherein:
Z is CR₁₆ or N; and
each R₁₆ and each R₁₇ is independently selected from H, halogen, alkyl, substituted alkyl, alkoxy, substituted alkoxy, hydroxy, cyano, nitro, aryl, substituted aryl, heterocycle, substituted heterocycle, heteroaryl, substituted heteroaryl, amino, substituted amino, carboxy, carboxy ester, sulfonyl, sulfonate, sulfonamide and substituted sulfonamide.

10. The method of claim 9, wherein the inhibitor is a compound of Table 4.

11. The method of claim 1, wherein R₃ and R₅-R₁₂ are selected from one of the following combinations:

| Combination | R₃ | R₅ | R₆ | R₇ | R₈ | R₉ | R₁₀ | R₁₁ | R₁₂ |
|---|---|---|---|---|---|---|---|---|---|
| 1 | H | H | H | H | H | H | CN | H | H |
| 2 | H | Br | H | H | H | H | CN | H | H |
| 3 | H | Cl | H | H | H | H | CN | H | H |
| 4 | H | Cl | H | H | H | Cl | CN | H | H |
| 5 | H | F | H | H | H | Cl | CN | H | H |
| 6 | H | F | H | H | H | Me | CN | H | H |
| 7 | H | Br | H | H | H | Cl | CN | H | H |
| 8 | H | F | H | H | H | H | CN | H | H |
| 9 | H | H | Br | H | H | H | CN | H | H |
| 10 | H | H | F | H | H | H | CN | H | H |
| 11 | H | H | Br | H | Cl | H | CN | H | H |
| 12 | H | H | Br | H | F | H | CN | H | H |
| 13 | H | H | Br | H | H | Cl | CN | H | H |
| 14 | H | H | H | Br | H | H | CN | H | H |
| 15 | H | Ph | H | H | H | H | CN | H | H |
| 16 | H | H | Cl | H | H | H | CN | H | H |
| 17 | H | H | CF₃ | H | H | H | CN | H | H |
| 18 | H | H | CN | H | H | H | CN | H | H |
| 19 | H | F | H | H | H | CN | Me | H | H |
| 20 | H | F | H | H | H | CN | F | H | H |
| 21 | H | F | H | H | H | Me | NO₂ | H | H |
| 22 | H | F | H | H | Cl | H | NO₂ | H | H |
| 23 | H | Cl | H | H | Cl | H | NO₂ | H | H |
| 25 | H | Cl | H | H | H | Me | NO₂ | H | H |
| 26 | H | F | H | H | H | Cl | Br | H | H |
| 30 | H | Cl | H | H | H | Cl | Br | H | H |
| 31 | H | F | H | H | H | Cl | Cl | H | H |
| 32 | H | F | H | H | H | F | F | H | H |
| 33 | H | F | H | H | H | F | H | H | H |
| 34 | H | F | H | H | H | Me | Cl | H | H |
| 35 | H | Cl | H | H | H | Me | Cl | H | H |
| 36 | H | F | H | H | Me | H | Cl | H | H |
| 37 | H | F | H | H | F | H | Cl | H | H |
| 38 | H | F | H | H | Cl | H | Cl | H | H |
| 39 | H | F | H | H | F | H | F | H | H |
| 40 | H | F | H | H | F | F | H | H | H |
| 41 | H | F | H | H | H | F | H | F | H |
| 42 | H | F | H | H | H | Cl | H | Cl | H |
| 43 | H | F | H | H | H | Cl | H | H | H |
| 44 | H | F | H | H | H | H | CF₃ | H | H |
| 45 | H | F | H | H | H | H | OCF₃ | H | H |
| 46 | H | F | H | H | H | Cl | OCF₃ | H | H |
| 47 | H | F | H | H | H | CF₃ | Cl | H | H |
| 48 | H | F | H | H | H | CF₃ | F | H | H |
| 49 | H | F | H | H | H | CF₃ | Me | H | H |
| 50 | H | F | H | H | H | CF₃ | H | H | H |
| 51 | H | F | H | H | H | CF₃ | H | CF₃ | H |
| 52 | H | F | H | H | H | OCF₃ | H | H | H |
| 53 | H | F | H | H | H | H | Ph | H | H |
| 54 | H | F | H | H | H | H | OMe | H | H |
| 55 | H | F | H | H | H | OMe | F | H | H |
| 56 | H | F | H | H | H | Me | F | H | H |
| 57 | H | F | H | H | H | H | CO₂Et | H | H |
| 58 | H | F | H | H | H | H | H | CO₂Et | H |
| 59 | H | F | H | H | H | H | H | NMe₂ | H |
| 60 | H | F | H | H | Cl | H | H | H | H |
| 61 | H | F | H | H | F | H | H | H | H |
| 62 | H | F | H | H | CF₃ | H | H | H | H |
| 63 | H | F | H | H | Me | H | H | CF₃ | H |
| 151 | H | Cl | H | H | H | Br | Cl | H | H |
| 152 | H | Cl | H | H | H | H | CF₃ | H | H |
| 153 | H | Cl | H | H | H | Cl | Br | H | CH₃ |
| 154 | H | Cl | H | H | H | H | CF₃ | H | CH₃ |
| 155 | H | Br | H | H | H | Br | Cl | H | H |
| 156 | H | Br | H | H | H | H | CF₃ | H | H |
| 157 | H | Br | H | H | H | Cl | Br | H | CH₃ |
| 158 | H | Br | H | H | H | H | CF₃ | H | CH₃ |
| 159 | H | Br | H | H | H | CF₃ | H | CF₃ | H |
| 160 | H | F | H | H | H | H | NO₂ | H | H |
| 161 | H | Cl | F | H | H | H | CN | H | H |

12. A method for inhibiting the proliferation of a cancer cell, in an individual in need thereof, the method comprising contacting a cancer cell with an effective amount of a CREB transcription inhibitor compound to inhibit proliferation of the cell.

13. The method of claim 12, wherein the cell is an Acute Myeloid Leukemia (AML) cell or an Acute Lymphomblastic Leukemia (ALL) cell.

14.-16. (canceled)

17. A CREB transcription inhibitor of formula (I): 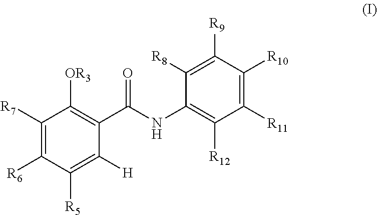
wherein:
R₃ is selected from H and a promoiety;
R₈, R₉, R₁₀, R₁₁ and R₁₂ are independently selected from H, halogen, alkyl, substituted alkyl, alkoxy, substituted alkoxy, an electron withdrawing group, phenyl, substituted phenyl, substituted amino, carboxy ester; and
R₅, R₆ and R₇ are independently selected from H, F, Cl, Br, alkyl, substituted alkyl, an electron withdrawing group, alkoxy and substituted alkoxy, wherein optionally R₆ and R₇ or R₅ and R₆ are cyclically linked to form a fused aryl or heteroaryl ring which is optionally further substituted;
or a salt thereof, or a prodrug form thereof.

18. The inhibitor of claim 17, wherein the inhibitor is a compound of one of Tables 1-4.

19. The method of claim 7, wherein the inhibitor is of the formula (XVIIIa).

20. The method of claim 19, wherein:
R₉ and R₁₁ are each trifluoromethyl;
R₅ is selected from Cl, Br and F; and
R₃, R₇-R₈, R₁₀, and R₁₂ are each H.

21. The method of claim 20, wherein R₅ is Cl.

22. The method of claim 20, wherein R₅ is Br.

23. The inhibitor of claim 17, wherein the inhibitor is of the formulae (XVIIIa): 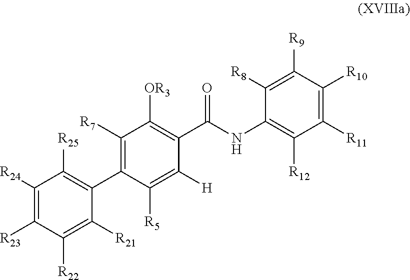
wherein R₂₁-R₂₅ are independently selected from H, halogen, alkyl, substituted alkyl, alkoxy, substituted alkoxy, hydroxy, cyano, nitro, aryl, substituted aryl, heterocycle, substituted heterocycle, heteroaryl, substituted heteroaryl, amino, substituted amino, carboxy, carboxy ester, sulfonyl, sulfonate, sulfonamide and substituted sulfonamide.

24. The inhibitor of claim 23, wherein:
R₉ and R₁₁ are each trifluoromethyl;
R₅ is selected from Cl, Br and F; and
R₃, R₇-R₈, R₁₀, and R₁₂ are each H.

25. (canceled)

26. (canceled)

27. The inhibitor of claim 17, wherein R³ is a promoiety selected from acyl, substituted acyl, phosphate ester, and [1,3]oxazine-2,4(3H)-dione.
